(19) United States
(12) Patent Application Publication
(10) Pub. No.: US 2015/0201918 A1
(43) Pub. Date: Jul. 23, 2015

(54) Surgical Handpiece

(71) Applicant: OSSEODYNE SURGICAL SOLUTIONS, LLC, Irvine, CA (US)

(72) Inventors: Ajay Kumar, Palmdale, CA (US); Jonathan A. Barney, Newport Beach, CA (US)

(73) Assignee: OSSEODYNE SURGICAL SOLUTIONS, LLC, Irvine, CA (US)

(21) Appl. No.: 14/569,699

(22) Filed: Dec. 13, 2014

Related U.S. Application Data
(60) Provisional application No. 61/923,038, filed on Jan. 2, 2014.

(51) Int. Cl. A61B 17/00; A61B 17/88; A61B 17/16

(52) U.S. Cl. CPC A61B 17/00; A61B 17/1628; A61B 17/8875; A61B 17/1624; A61B 2017/00402; A61B 2017/00734; A61B 2017/00725; A61B 2019/4836

(57) ABSTRACT

Embodiments of the present invention provide a unique surgical handpiece having improved operation, durability and reliability. In one embodiment, the present invention provides a motorized handheld surgical instrument having one or more sensors for sensing motion, position, pressure, humidity, and various other environmental conditions relevant to the operation and maintenance of the surgical instrument.

### BACKGROUND

1. Field of the Invention

The present invention generally relates to motorized handheld devices and, in particular, to motorized handheld surgical instruments having one or more rotating and/or reciprocating working elements.

2. Description of the Related Art

Powered surgical instruments, or “handpieces” as they are commonly known, are specialized tools that are commonly used in many medical specialties to drive surgical blades, drills, taps, drivers, cutting instruments and various other rotating and/or reciprocating working elements. These specialized tools are typically used for performing various diverse functions, including, resection, comminution, dissection, debridement, shaving, drilling, tapping, pulverizing, and shaping of anatomical tissues. Powered surgical handpieces and similar motorized surgical tools may also be used for driving or inserting medical screws, dental implants, pins and staples into the human body. Handpieces for general surgical purposes may be configured to selectively couple to and drive a variety of different surgical instruments designed to perform one or more specialized procedures.

Typical surgical handpieces may be externally or internally powered. An externally-powered handpiece typically includes a control console and a flexible cable that connects the handpiece to the console. The control console is typically configured to selectively activate and/or control the amount of energy or power delivered to an electric, hydraulic or pneumatic motor disposed within the powered surgical handpiece. An internally-powered handpiece typically includes an electric motor and one or more internally-contained replaceable and/or rechargeable batteries and associated control circuitry configured to selectively activate and/or control the amount of energy or power delivered to the electric motor.

Powered surgical instruments can provide significant speed advantages over manual tools in completing various surgical tasks. However, they can sometimes introduce new problems and challenges such as decrease or lack of precision control, unreliable operation over time and/or unpredictable failure modes. Another particularly difficult challenge with almost all powered surgical instruments is ensuring long-term survivability and reliable operation through multiple surgical procedures and repeated cycles of autoclaving or sterilization. Autoclaving is a form of sterilization which involves exposing an entire device to high-temperature steam and alternating cycles of high and low pressure. The autoclaving process creates a hostile environment specifically designed to kill any bacteria, viruses, fungi, and spores that may be present. Repeated exposure to this hostile environment can significantly shorten the useful life expectancy of surgical devices, especially powered surgical instruments having internal electric motors and other sensitive electronic components.

### SUMMARY

Embodiments of the present invention provide a unique surgical handpiece having improved operation, durability and reliability.

In one embodiment the present invention provides a motorized handheld surgical instrument having one or more rotating and/or reciprocating working elements and wherein one or more motion or position sensors are provided and configured to enable a user to control some or all of the functions of the surgical instrument through one or more motions or gestures (such as turning, twisting, torquing, pushing or pulling) imparted by a hand of a user on the surgical instrument. In another embodiment sensed motions or gestures may include, for example and without limitation: i) full-body translational or rotational movement of the handpiece, ii) absolute or relative position, angle, or orientation of the handpiece in free space, iii) amount of input torque and/or force applied by a user to the handpiece, iv) amount of reaction torque and/or force applied by a user in resistance to translational or rotational movements of the handpiece, or v) tapping, jerking, shaking, or dropping the handpiece.

In another embodiment the present invention provides a motorized handheld surgical instrument having one or more humidity or pressure sensors configured to provide control or diagnostics feedback to an internal control system or performance monitoring system of the surgical instrument.

In another embodiment the present invention provides a motorized handheld surgical instrument wherein the traditional metallic handpiece housing is replaced with a housing fabricated from a highly-thermally-conductive polymer-based composite. In accordance with another embodiment, an inner housing is provided comprising a thermally-conductive sleeve or heat sink disposed within or immediately adjacent to an annular cavity formed between the outer housing and the motor. In accordance with another embodiment, the thermally-conductive sleeve is formed from a highly-thermally-conductive polymer-based composite materials. In another embodiment, the thermally-conductive sleeve is separately formed from a CNT-enhanced metallic matrix material.

In another embodiment the present invention provides a motorized handpiece having one or more motor or drive train elements configured to transmit torque or other mechanical forces through a wall of a sealed housing via a magnetic coupling. In another embodiment the present invention provides a motorized handpiece having a modified motor wherein an external rotor magnetically communicates torque with a fixed internal stator through the walls of a sealed vessel.

In another embodiment the present invention provides a motorized handpiece having an improved keypad design comprising an integrally molded silicone upper portion and one or more flexibly suspended depressible buttons. In another embodiment the keypad may be formed partially or entirely from pressure-sensitive conductive rubber. In another alternative embodiment, the keypad may be formed partially or entirely from a compressible dielectric material sandwiched between one or more conductive plates. In another alternative embodiment, the keypad may include one or more touch sensor elements or other solid-state electronic switches activated by human touching and/or pressing of a finger. In another alternative embodiment, the keypad may comprise one or more solid-state piezoelectric input devices or piezo switches.

In another embodiment the present invention provides a motorized handpiece wherein one or more condition sensors are provided for sensing various operating and non-operating (e.g., during autoclaving) conditions relevant to the handpiece, such as heat, temperature, pressure, moisture, humidity, leakage, and the like. In another embodiment, sensor data from these and/or any other sensors may be used for purposes of providing improved feedback control and/or for purposes of providing improved performance and maintenance monitoring. In accordance with one embodiment, sensor data may be used to provide early failure detection and/or recommended testing, inspection or maintenance of a motorized handpiece.

In another embodiment the present invention provides a motorized handpiece wherein one or more condition sensors are provided for sensing various operating and non-operating (e.g., during autoclaving) conditions relevant to the handpiece, and wherein the resulting sensor data is monitored and/or recorded for purposes of providing improved performance, durability, maintenance monitoring, and failure prediction. In accordance with another embodiment multiple redundant sensors are preferably provided at various locations within the handpiece housing in a multi-redundant fault-tolerant design.

In another embodiment the present invention provides a motorized handpiece wherein a dynamic CMS and maintenance protocol is deployed, such that the actual usage or system degradation is taken into account and the required maintenance intervals are regularly updated or even fully determined during the service life. In another embodiment a user-alert system is provided incorporating a pair of LED indicators configured to shine through a sealed window for alerting a user to a detected fault condition.

The attached figures and accompanying disclosure illustrate and describe multiple embodiments of various powered surgical instruments having features and advantages of the invention as more-fully described herein. All of these embodiments are intended to be within the scope of the invention herein disclosed. These and other embodiments and obvious variations of the present invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

Having thus summarized the general nature of the invention and its essential features and advantages, certain preferred embodiments and obvious modifications thereof will become apparent to those skilled in the art from the detailed description herein having reference to the figures that follow, of which:

FIG. 1 is a perspective view of a surgical handpiece having features and advantages in accordance with one embodiment of the present invention;

FIG. 2 is a partial-sectioned simplified schematic view of a surgical handpiece having features and advantages in accordance with another embodiment of the present invention;

FIG. 3 is a graph which shows the relative density and thermal conductivity of a CNT/Cu metallic matrix material containing varying amounts of CNT as a volume fraction;

FIG. 4 is a process schematic illustrating how dendritic copper particles are gradually deformed through mechanical impact into spheres, how agglomerated CNT clumps are disintegrated into individual CNTs and how CNTs embed into the outer surface of each spherical copper particle to form a composite CNT/Cu particle;

FIG. 5 is a partial schematic diagram of a battery-operated motorized handpiece illustrating major sources of potential leakage;

FIG. 6A is a partial-sectional view of a battery-operated motorized handpiece illustrating major sources of potential leakage;

FIG. 6B is a partial-sectioned detailed schematic view of a drivetrain sealing interface of a surgical handpiece having features and advantages in accordance with the present invention;

FIGS. 7A-D are schematic and partial-sectional views of a magnetic coupler configured for use in a surgical handpiece having features and advantages in accordance with another embodiment of the present invention;

FIGS. 8A and 8B are schematic and partial-sectional views of a modified brushless DC motor configured for use in a surgical handpiece having features and advantages in accordance with another embodiment of the present invention;

FIGS. 9A and 9B are finite-element stress analysis diagrams of a conventional silicone-molded keypad button subjected to positive (FIG. 9A) and negative (FIG. 9B) pressure cycles during autoclaving;

FIG. 10A is a partial-sectional view of an improved keypad design configured for use in a surgical handpiece having features and advantages in accordance with another embodiment of the present invention;

FIG. 10B is a partial-exploded view of an improved keypad design configured for use in a surgical handpiece having features and advantages in accordance with the present invention;

FIG. 10C is a sectional detail view of an improved keypad interface with integrally molded button caps configured for use in a surgical handpiece having features and advantages in accordance with the present invention;

FIG. 10D is a partial-sectional detail view of an improved keypad button configured for use in a surgical handpiece having features and advantages in accordance with the present invention;

FIG. 11 is a bottom plan view of an improved keypad cover plate design having features and advantages in accordance with the present invention;

FIGS. 12A and 12B are schematic sectional views of a pressure-sensitive conductive rubber configured for use in a surgical handpiece having features and advantages in accordance with another embodiment of the present invention and illustrating the basic principles of operation thereof;

FIGS. 13A and 13B are schematic views of a pressure-sensitive variable capacitor configured for use in a surgical handpiece having features and advantages in accordance with another embodiment of the present invention;

FIGS. 14A and 14B are schematic assembly and partial sectional views of an improved stepped quad-O-ring contact pin sealing system configured for use in a surgical handpiece having features and advantages in accordance with another embodiment of the present invention;

FIG. 15 is a schematic partial-sectional view of a surgical handpiece having one or more internally-disposed accelerometer sensors and gyro sensors in accordance with another embodiment of the present invention;

FIG. 16 is a partial perspective view of a surgical handpiece having one or more tap- or pressure-sensitive virtual buttons in accordance with another embodiment of the present invention;

FIG. 17 is a graph illustrating the time domain response of an accelerometer responding to sensed tapping on the housing of a surgical handpiece having features and advantages in accordance with another embodiment of the present invention;

FIG. 18 is a schematic partial sectional view of a surgical handpiece having one or more internal pressure sensors disposed at or near the drive train in accordance with another embodiment of the present invention;

FIG. 19 is a schematic partial sectional view of a surgical handpiece having one or more internal pressure sensors disposed in a posterior cavity in accordance with another embodiment of the present invention;

FIG. 20 is a schematic sectional view of a pressure sensor comprising a pressure-sensing flexible membrane configured for use in a surgical handpiece having features and advantages in accordance with another embodiment of the present invention;

FIG. 21 is an electrical schematic diagram of a detection and signal conditioning circuit configured for use in a surgical handpiece having features and advantages in accordance with another embodiment of the present invention;

FIG. 22 is a partially exploded sectional view of a pressure sensor comprising a pressure-sensing flexible membrane configured for use in a surgical handpiece having features and advantages in accordance with another embodiment of the present invention;

FIGS. 23A and 23B are lateral and longitudinal cross-sectional views of a sensor containment and isolation vessel configured for use in a surgical handpiece having features and advantages in accordance with another embodiment of the present invention;

FIG. 24 is a schematic partial perspective view of a surgical handpiece having one or more internal humidity sensors in accordance with another embodiment of the present invention;

FIG. 25 is a schematic detail view of a thermal-conductivity-based humidity sensor configured for use in a surgical handpiece having features and advantages in accordance with another embodiment of the present invention;

FIG. 26 is a schematic detail view of a CMOS-based humidity sensor configured for use in a surgical handpiece having features and advantages in accordance with another embodiment of the present invention;

FIG. 27 is a schematic electrical diagram illustrating one possible embodiment of a signal processing circuit suitable for use with the humidity sensor of FIG. 26;

FIG. 28 is a graph of measured axial load in pounds force applied to a bone-penetrating pin by six different surgeons in a clinical study;

FIG. 29 is a graph of measured insertion torque applied to a bone-penetrating screw versus the number of screw rotations;

FIG. 30 is a schematic block diagram of one embodiment of a system-level redundancy architecture for an inertial navigation system in which three different sensor groups provide inertial input data to a fault-tolerant control management system configured for use in a surgical handpiece having features and advantages in accordance with another embodiment of the present invention;

FIG. 31A is a schematic block diagram of another embodiment of a system-level redundancy architecture incorporating a central data fusion filter and feedback correction configured for use in a surgical handpiece having features and advantages in accordance with another embodiment of the present invention;

FIG. 31B is a schematic block diagram of a more generalized embodiment of a system-level redundancy architecture incorporating a central data fusion filter, feedback correction and multiple redundant sensor systems configured for use in a surgical handpiece having features and advantages in accordance with another embodiment of the present invention;

FIG. 32 is a schematic block diagram of a more sophisticated embodiment of a system-level redundancy architecture incorporating a federated data fusion filter, feedback correction and multiple redundant sensor systems configured for use in a surgical handpiece having features and advantages in accordance with another embodiment of the present invention;

FIG. 33 is a graph of a typical P-F or “Moubray” curve used to model or predict failure patterns;

FIGS. 34A and 34B are graphs illustrating a model for comparing or measuring the performance of a CMS by analyzing and comparing two interrelated parameters, γ (probability of detection) and η (efficiency of detection);

FIG. 35 is a schematic block diagram of a performance trending algorithm configured for use in a surgical handpiece having features and advantages in accordance with another embodiment of the present invention;

FIG. 36 is a graph of a P-F curve based on modeling the deterioration process of a typical motorized handpiece over the course of its useful life;

FIG. 37 is a graph illustrating how predictive trending can be used to predict future system performance based on observed past system performance;

FIGS. 38A and 38B are partial perspective views of one embodiment of a user-alert system incorporating a pair of LED indicators configured to shine through a sealed window for alerting a user to a detected fault condition; and

FIG. 39 is a simplified electrical schematic of one embodiment of a microprocessor-controlled user-alert system incorporating a pair of LED indicators.

For convenience of description and for better clarity and understanding of the invention, similar elements in different figures may be identified with similar or even identical reference numerals. However, not all such elements in all embodiments are necessarily identical as there may be differences that become clear when read and understood in the context of each particular disclosed preferred embodiment.

### DETAILED DESCRIPTION

### Definitions

The following terms as used herein in the specification and in the claims shall be defined and understood as follows, regardless of any other ordinary or understood meanings, dictionary definitions, definitions in documents incorporated by reference, or other possible meanings of the defined terms:

The indefinite articles “a” and “an” should be understood to mean at least one.

The conjunctive phrase “and/or” should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “communicatively coupled” or “communicative coupling” means that there is a path or channel of communication from one component to another, whether the path is direct or indirect and whether such path includes a path through one or more intervening components.

The phrase “electrically coupled” or “electrical coupling” means that there is an electrical current, voltage or signal path from one component to another, whether the path is direct or indirect and whether such path includes a path through one or more intervening components.

The conjunctive article “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

### Acronyms

For the convenience of the reader certain acronyms and abbreviations used herein in the specification and claims are listed below:

“ASIC” application-specific integrated circuit

“CBM” condition-based maintenance

“CMS” condition monitoring systems

“CNT” carbon nanotube

“CTE” coefficient of thermal expansion

“DLC” diamond-like carbon

“EMF” electro-motive force

“HDPE” high-density polyethylene

“INS” inertial navigation system

“IT” insertional torque

“LCD” liquid crystal display

“LED” light-emitting diode

“LTCC” low-temperature co-fired ceramic

“MEMS” micro-electro-mechanical system

“MWCNT” multi-walled carbon nanotubes

“NEMS” nano-electro-mechanical system

“OLED” organic light-emitting diode

“PCB” printed circuit board

“PEEK” polyether ether ketone

“PMMA” polymethylmethacrylate

“PS” polystyrene

“PZT” lead zirconate-titanate (Pb(Zrl-xTix)03)

“RH” relative humidity

“SOIC” small-outline integrated circuit

“ST” stripping torque

“SWCNT” single-walled carbon nanotubes

### Basic Construction

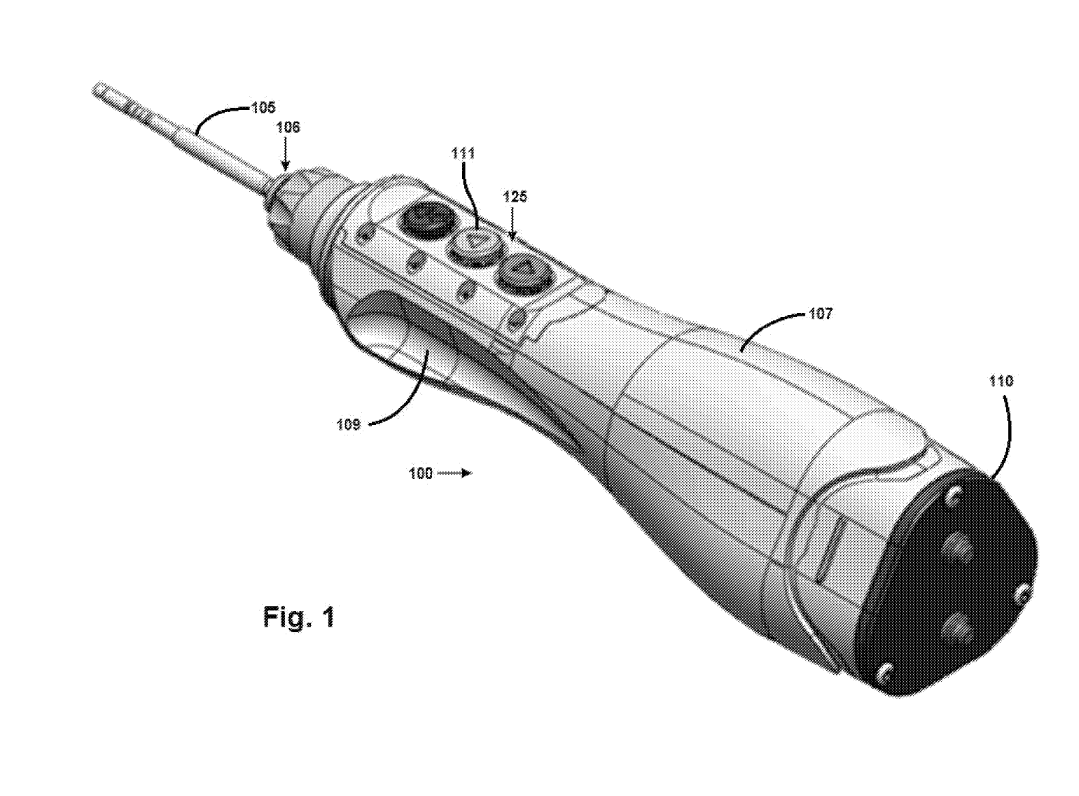

FIG. 1 is a perspective view of a surgical handpiece 100 having features and advantages according to one embodiment of the present invention. The handpiece 100 generally comprises an outer housing 107 configured to be comfortably gripped in a single hand of a surgeon and selectively manipulated to guide a rotating and/or reciprocating working element 105, such as a surgical blade, drill, tap, driver, or cutting instrument. The outer housing 107 may comprise one or more optional surface contours 109 configured to provide an ergonomic handle or gripping surface suitable for comfortably gripping and operating the device.

The housing 107 may include a keypad 125 comprising multiple input buttons 111, as shown. For example, the keypad 125 may allow a user to control certain handpiece functions such as basic motor controls (e.g., on/off, forward/reverse, speed, torque, etc.) and various other functions as may be expedient or desired. Various output indicators (e.g., LED lights, OLED or LCD display elements, buzzers, vibration generators, and the like) may also be provided as part of the overall user interface for safely, reliably and precisely controlling and operating the handpiece 100 as described in more detail herein.

The entire housing 107 is preferably sealed so as to substantially prevent ingress of water, debris and other potential contaminants. See, for example, U.S. Patent Application 2010-0102517A1 to Kumar, the entire contents of which is incorporated herein by reference. Alternatively, the housing may be partially sealed and partially vented. A removable cover or end cap 110 may be provided and sealed against the outer housing 107. Preferably, the end cap 110 is configured to provide periodic access to an internal cavity that contains one or more serviceable or replaceable components such as, for example, replaceable circuit boards.

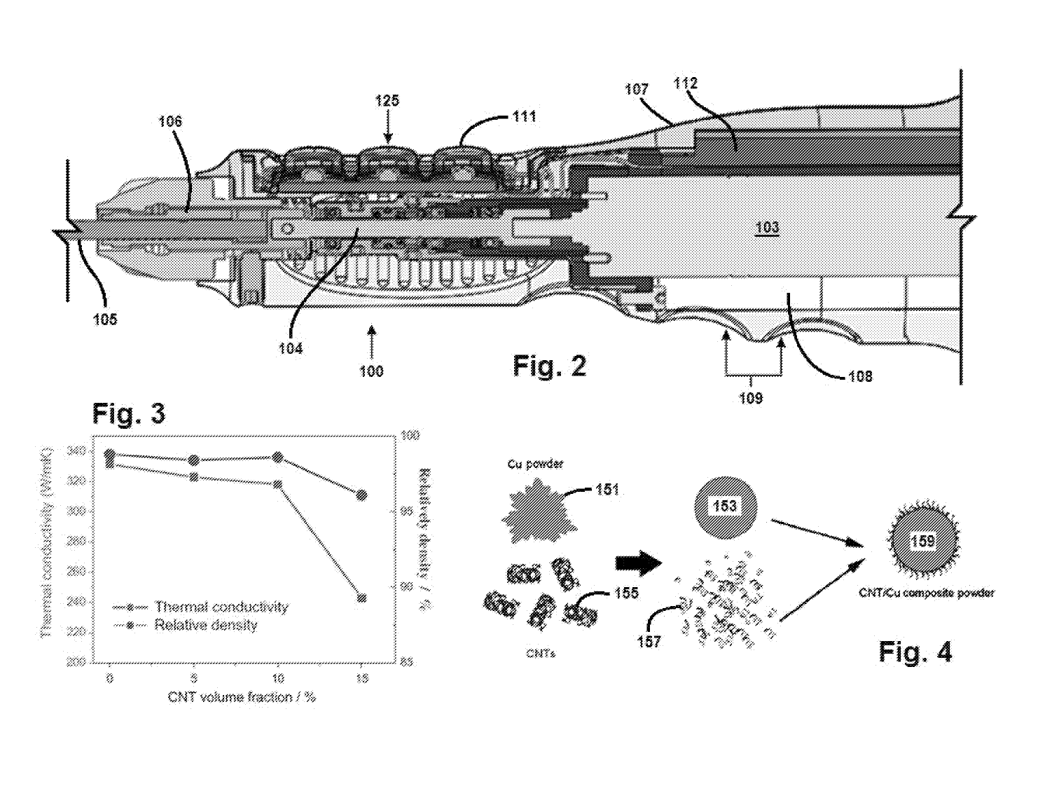

FIG. 2A is a partial-sectioned simplified schematic view of a surgical handpiece 100 having features and advantages according to another embodiment of the present invention. The handpiece 100 generally includes an electric motor 103 configured to drive a rotating and/or reciprocating working element 105, such as a surgical blade, drill, tap, driver, or cutting instrument. Preferably the electric motor 103 is of a sealed type so as to substantially prevent ingress of water, debris and other potential contaminants. See, for example, U.S. Patent Application 2006-0006094A1 to Hofmann, the entire contents of which is incorporated herein by reference.

The electric motor 103 is coupled to a drive shaft 104 which, in turn, is coupled to a chuck or collet 106 that is configured to detachable interface with the working element 105. Alternatively, or in addition, persons skilled in the art will readily appreciate that one or more intermediary transmission or converter devices (not shown) may be interposed between the electric motor 103 and the chuck or collet 106 in order to provide a desired step-up or step-down in output rotational speed or torque of the working element 105, to change the axis of rotation of the working element 105 (e.g., a right-angle transmission device), and/or to convert rotational movement of the motor 103 into translating or reciprocating movement (or other movements), as desired. Alternatively, the motor 103 may comprise an electro-magnetic solenoid or reciprocating linear magnetic motor (now shown) where it is desired to directly provide translating or reciprocating movement without an intermediary transmission or converter device. Those skilled in the art will readily appreciate that any rotating or reciprocating elements in the handpiece 100 are preferably statically and/or dynamically balanced or/or counterbalanced so as to reduce or minimize any undesired vibrations of the device.

As illustrated in FIGS. 1 and 2A, the outer housing 107 is configured to receive and support the motor 103 and associated internal components. The outer housing 107 also preferably provides an optional user-manipulable handle or gripping surface 109 suitable for comfortably gripping and operating the device. The entire housing 107 is preferably sealed so as to substantially prevent ingress of water, debris and other potential contaminants. An air gap 108 may be provided within the housing 107 forming an annular cavity surrounding the motor 103 for providing ventilation and cooling of the motor 103. One or more optional fans or other air-circulating elements (not shown) may also be provided and may be driven by either the motor 103 and/or by other means as desired. Optionally, the handpiece housing 107 immediately adjacent to or surrounding the air gap 108 may include one or more removable portions configured to facilitate cleaning or removal of debris or other contaminants.

### Outer Housing

Heat management is always a key consideration for any surgical handpiece design due to the presence of an internal electric motor (a primary heat source) and various associated electrical components inside a sealed or partially-sealed cavity. Traditional designs have typically employed a dielectric-coated sealed metallic housing and various solid heat sinks, conductive gels, and the like disposed between the housing and the motor in order to help dissipate heat away from the handpiece motor and other heat-producing components. See, for example, U.S. Patent Application US2011-0213395 to Corrington, the entire contents of which is incorporated herein by reference. However, this traditional design approach suffers from at least two major shortcomings:
1. the resulting handpiece housing is extremely heavy as it must have sufficient mass and volume not only to conduct and dissipate heat, but also to withstand drops without fracturing or suffering other physical damage; and
2. the dielectric coating tends to get removed and the seals tend to get degraded over time due to normal use and abuse, strong cleaning solutions, and multiple heat and pressure cycles encountered in the autoclaving or sterilization processes.

To overcome these and other shortcomings, one embodiment of the present invention replaces the traditional metallic handpiece housing with a housing fabricated from a highly-thermally-conductive polymer-based composite. Key advantages here include: lighter weight, improved durability, corrosion resistance, faster/easier fabrication, and lower manufacturing cost.

Fundamentally, heat transfer involves the transport of energy from one place to another by energy carriers. In a gas phase, gas molecules carry energy either by random molecular motion (diffusion) or by an overall drift of the molecules in a certain direction (advection). In liquids, energy can be transported by diffusion and advection of molecules. In solids, phonons, electrons, or photons transport energy. Phonons are quantized modes of vibration that occur in a rigid crystal lattice. These are the primary mechanism of heat conduction in most polymers since free movement of electrons is typically very low in polymer materials. In accordance with theoretical prediction, the Debye equation (below) is typically used to calculate the thermal conductivity of polymers as follows:

[symbol]

=(Cpvl)/3

where:

λ=thermal conductivity of thermal conductivity of the polymer

Cp=the specific heat capacity per unit volume;

v=the average phonon velocity; and

l=the phonon mean free path.

For amorphous (i.e., non-crystalline) polymers, l is an extremely small constant (e.g., a few angstroms) due to phonon scattering from numerous defects, leading typically to a very low thermal conductivity for most amorphous polymers. Crystallinity strongly affects thermal conductivity characteristics of polymers, which typically varies from 0.2 W/mK for completely amorphous polymers such as polymethylmethacrylate (PMMA) or polystyrene (PS), to 0.5 W/mK for highly crystalline polymers such as high-density polyethylene (HDPE). The thermal conductivity of semi-crystalline polymers generally increases with crystallinity. The thermal conductivity of an amorphous polymer increases with increasing temperature to the glass transition (Tg), while it decreases above Tg.

It is well know that the thermal conductivity of most polymers can be enhanced by the addition of thermally conductive fillers, including graphite, carbon black, carbon fiber, ceramic or metal particles. Filler loadings of 5-10% by volume, and more preferably 30% or more, can be used to achieve sufficient thermal conductivity (e.g., higher than about 4 W/mK). Carbon-based fillers such as graphite, carbon fiber and carbon black are well-known fillers that can be used to enhance thermal conductivity in a wide variety of polymer-based materials. Graphite is particularly preferred in one embodiment because of its good thermal conductivity, low cost and fair dispersability in a polymer matrix.

In accordance with another embodiment, the handpiece housing 107 is fabricated from a thermally conductive polymer-based composite material prepared by the incorporation of one or more metallic particles. Incorporation of powdered metallic filler in a polymer matrix may result in both an increase in thermal conductivity and electrical conductivity. However, a density increase is also obtained when adding significant metal loadings to the polymer matrix, which can limit its use in certain applications where lightweight is a priority. Metallic particles used for thermal conductivity improvement include powders of aluminum, silver, copper and nickel. Polymers that can be modified with the inclusion of metallic particles include polyethylene, polypropylene, polyamide, polyvinylchloride and epoxy resins.

For polymers incorporating heat-conducting fillers (either metallic or non-metallic), overall thermal conductivity performance will depend on the thermal conductivity of the cured polymer resin, the particular filler material used, the particle shape and size, the volume fraction of filler material used, and the spatial arrangement of the filler material in the polymer matrix. The upper theoretical bound of thermal conductivity kc for a composite material is typically calculated by the parallel mixture model according to following equation:

kc=kpφp+kmφm

where kc, kp, km are the thermal conductivity of the composite, particle, matrix, respectively, and φp, φm are the volume fractions of particles and matrix, respectively. The parallel mixture model maximizes the contribution of the conductive filler material by implicitly assuming perfect contact between adjacent particles in a fully percolating network. This assumption may not necessarily hold in practice and will vary significantly depending on the particular polymer and filler material used. Generally, smaller and more uniformly sized/shaped particles will result in increased contact and greater thermal conductivity in a polymer-based composite matrix.

In accordance with another embodiment, the handpiece housing 107 is fabricated from a thermally conductive polymer-based nanocomposite material prepared by the incorporation of one or more thermally conductive fillers including at least a substantial portion by volume of carbon nanotubes (CNT). The outstanding thermal conductivity of CNT and its superior mechanical properties provide particularly attractive advantages in the context of a handpiece housing. Added as a reinforcement material to a polymer-based resin (e.g. polyester, vinyl ester, epoxy, phenolic, polyimide, polyamide, polyethylene, polymethyl-methacrylate, polypropylene, PEEK), CNTs provide a basic building block for producing many advanced engineering composite materials having unprecedented mechanical and thermal properties, including ultra-high elastic modulus (˜1 TPa), high tensile strength (˜150 GPa), high thermal conductivity (3000-6000 W/mK), and a low coefficient of thermal expansion (2.7×10⁻⁶/K to 4.4×10⁻⁶/K). By appropriately selecting, adjusting and varying the chemical composition and structural make-up of various CNT-reinforced materials as taught and described herein, the above-noted material properties can be enhanced and optimized for purposes of providing an improved surgical handpiece 100 or other motorized handheld device having desired features and advantages in accordance with the present invention.

There are two main kinds of carbon nanotubes relevant to the present invention: single-walled carbon nanotubes (SWCNTs) comprising individual cylinders 1-2 nm in diameter and made up of a single rolled graphene sheet, and multi-walled carbon nanotubes (MWCNTs) comprising a multi-layered structure made up of several concentric graphene cylinders, with weak Van der Waals forces binding the inner and outer tubes together. SWCNTs are significantly smaller in diameter compared to MWCNTs and the thermal properties may differ significantly. MWCNTs consist of nested graphene cylinders coaxially arranged around a central hollow core with interlayer separations of about 0.34 nm, similar to the interplane spacing of graphite. MWCNTs are often curled, kinked and some of them are highly twisted with each other forming big CNT bundles having strong inter-tube van der Waals attraction.

Carbon nanotubes are exceptionally good thermal conductors along the axial direction of the tube, but moderate to poor thermal conductors in directions lateral to the tube axis. Measurements show that a SWCNT has a room-temperature thermal conductivity along its axis of about 3500 W/mK. This compares very favorably even to copper, a metal well known for its good thermal conductivity, which transmits only about 385 W/mK at room temperature. A SWCNT has a room-temperature thermal conductivity across its axis (in the radial direction) of about 1.52 W/mK, which is about as thermally conductive as porcelain. MWCNTs can have even higher thermal conductivities, depending on the particular chemical composition and structural makeup.

The transport of thermal energy in CNTs is believed to occur via a phonon conduction mechanism. The phonon conduction in nanotubes is influenced by several processes, including the number of phonon active modes, the boundary surface scattering, the length of the free path for the phonons. Because CNT has ultra-high conductivity along the tube or axial direction but low conductivity in radial directions, the random distribution of CNTs within a polymer matrix would result in various tube orientations which could limit the unidirectional heat transfer mechanism, reducing overall conductivity of the composite material. Processing the uncured or partially-cured CNT-laden polymer material in a manner that encourages or promotes the arrangement and axial alignment of CNTs along a desired heat transfer vector can improve the vector-specific conductivity of the resulting composite material. For example, processing the uncured or partially-cured CNT-laden polymer by pulling or stretching the material (either once or repeatedly) and/or by pulling or stretching filaments of material as it is extruded from a die is one effective method for encouraging a desired arrangement or alignment of CNTs.

The phonon mean free paths are relatively long in nanotubes: 500 nm for a MWCNT and even longer for a SWCNT. It is well known that CNTs are characterized by a large aspect ratio (length divided by diameter) and a very large surface area for a given volume of material. Diameter and length are two key parameters to describe CNTs and directly affect the thermal conductivity of both CNTs and composites containing CNTs.

The thermal conductivity of SWCNTs is generally higher due to smaller diameters. The thermal conductivity of MWCNT at room temperature increases as diameter decreases (e.g., as outer walls are removed), varying from about 500 W/mK for an outer diameter of 28 nm to about 2069 W/mK for a 10 nm diameter. For length parameters (L) from 5 to 350 nm, the calculated thermal conductivity increases with increasing tube length and follows a La law, with a values between 0.54 (100 nm<L<350 nm) and 0.77 (L<25 nm).

In a polymer nanocomposite material, the large surface area of the nanoparticles promotes heat transfer due to the relatively large number of contact points between particles at the boundaries of the polymer/particle interfacial area. Scattering of phonons (which inhibits heat transfer) in a nanocomposite material is primarily due to the existence of interfacial resistance at the polymer/particle interfacial area. In a simplified model, the transmission of a phonon between two different materials (e.g., a CNT contained within a polymer matrix) depends on the existence of one or more common vibration frequencies between the two materials. The more closely matched the vibration frequencies (e.g., the more similar the materials are in elastic modulus), the more efficiently phonons can be transmitted. Another source of interfacial resistance is the imperfect physical contact between CNT and the polymer matrix within which it is contained. This primarily depends on surface wettability. Thus, to achieve optimal thermal conductivity in a nanocomposite material, it is desirable to have good thermal contact (i.e., low thermal resistance) between nanoparticle and polymer.

Most preferably, the housing 107 is formed at least in part from a polymer-based nanocomposite material comprising MWCNT@SiO₂/epoxy composite. This particular nanocomposite material has been demonstrated to have a combination of good thermal conductivity, electrical insulating properties and excellent mechanical properties. See, for example, Jin Gyu Park, Qunfeng Cheng, Jun Lu, Jianwen Bao, Shu Li, Ying Tian, Zhiyong Liang, Chuck Zhang, Ben Wang, “Thermal conductivity of MWCNT/epoxy composites: The effects of length, alignment and functionalization,” Carbon, Volume 50, Issue 6, May 2012, Pages 2083-2090, incorporated herein by reference in its entirety.

Advantageously, MWCNT@SiO₂ fillers have improved dispersibility when used within an epoxy matrix due to the role of the silica shell which helps avoid tube-tube contacts, tangling and bundling of the nanotubes. The silica shell on MWCNT@SiO₂ also serves as an intermediate transition layer between the relatively-soft epoxy matrix and the relatively-stiff CNT. For example, the modulus of elasticity of SiO₂ is 70 GPa, which between the 600-1000 GPa elasticity modulus of CNT and the 3 GPa elasticity modulus of epoxy. As a result, the less stiff silica shell on the MWCNT alleviates the modulus mismatch between the relatively-stiff MWCNTs and the relatively-soft epoxy matrix, thus greatly improving conduction of phonons and heat energy.

Epoxy/MWCNT@SiO₂ nanocomposites also advantageously retain relatively high electrical insulating properties. See, for example, Wei Cui, Feipeng Du, Jinchao Zhao, Wei Zhang, Yingkui Yang, Xiaolin Xie, Yiu-Wing Mai, “Improving thermal conductivity while retaining high electrical resistivity of epoxy composites by incorporating silica-coated multi-walled carbon nanotubes,” Carbon, Volume 49, Issue 2, February 2011, Pages 495-500, ISSN 0008-6223. For typical MWCNT nanocomposite materials, adding only 0.5 wt % of MWCNTs with graphite-like structure (i.e., no insulating shell) to epoxy sharply decreases the volume electrical resistivity compared to neat epoxy by ≈6 orders of magnitude. Further increasing CNT loading to 1 wt. % only decreases the electrical resistivity of the composite material very mildly, indicating that a percolating electron network is formed at CNT loadings less than 0.5 wt. %. However, the silica shell on the MWCNT@SiO₂ fillers is electrically insulating, thus advantageously increasing the tunneling energy barrier and limiting the intertube charge transport. Therefore, the epoxy/MWCNT@SiO₂ composites maintain almost the same volume electrical resistivity as the neat epoxy resin, but with significantly higher thermal conductivity. For example, with a 1 wt. % filler loading, the electrical resistivity of epoxy/MWCNT@SiO₂ composites decreases only slightly to 6.9×10¹⁴ Ωm compared to 1.5×10¹⁵ Ωm of neat epoxy.

The combination of high thermal conductivity and low electrical conductivity make MWCNT@SiO₂/epoxy nanocomposites a particularly desirable choice for handheld electrical appliances used in medical and surgical applications. Of course, those skilled in the art will readily appreciate that MWCNT@SiO₂ fillers can also be used with a wide variety of other polymer materials such as those disclosed and described herein. Similarly, those skilled in the art will also readily appreciate that a wide variety of other metallic-oxide-coated MWCNTs may be used instead of or in addition to MWCNT@SiO₂, including without limitation, those containing one or more of the following metal oxides: TiO₂, Ti2O₃, ZnO, WO₃, Fe₂O₃, SnO₂, CeO₂, Al₂O₃, ZrO₂, V₂O₄ and Er₂O₃.

### Inner Housing

As noted above, heat management is always a key consideration in a surgical handpiece design due to the presence of an internal electric motor (a primary heat source) and various associated electrical components all contained within a sealed or partially-sealed cavity. Typically, various solid heat sinks and/or conductive gels are disposed between the housing and the motor in order to help dissipate heat away from the motor and/or other heat-producing components. See, for example, U.S. Patent Application US2011-0213395 to Corrington. Alternatively, some ambient ventilation can be provided within a partially sealed housing, as disclosed and described above in connection with FIG. 2.

Another important consideration in surgical handpiece design is minimizing thermal expansion and, in particular, minimizing dissimilarities in the coefficient of thermal expansion (CTE) between one or more mating components subjected to thermal loading under design conditions (both during use and during sterilization/autoclaving). Large expansion coefficients and significant dissimilarities of expansion coefficients of mechanical components subjected to thermal loading can lead to thermal stress, thermo-mechanical fatigue and cracking or degrading of mechanically interfacing components during multiple autoclaving cycles and/or under heavy-use conditions. Materials with a relatively low CTE are particularly preferred when thermal loading under design conditions is expected to be high and/or when a device must withstand a large number of thermal loading cycles.

In accordance with one embodiment of the invention, an inner housing is provided comprising a thermally-conductive sleeve 112 or heat sink disposed within or immediately adjacent to the annular cavity 108 formed between the outer housing 107 and the motor 103. The thermally-conductive sleeve 112 is preferably formed from a metal-based material and/or one or more of the highly-thermally-conductive polymer-based composite materials disclosed and described above. The thermally-conductive sleeve 112 may also be formed either separately from or integrally with the outer housing 107. A thermally conductive adhesive or gel may be used, as desired, to provide a thermally conductive interface and/or mechanical bond between the thermally-conductive sleeve 112, the motor 103, and/or the outer housing 107.

In another embodiment, the thermally-conductive sleeve 112 is separately formed from a CNT-enhanced metallic matrix material. For example, various studies have revealed that the addition of 10-15 vol. % CNTs to aluminum can reduce the CTE of the resulting Al matrix material by as much as 65%, while only moderately reducing thermal conductivity. Studies have also shown similarly favorable results for copper, which has a CTE 30% lower than aluminum. For example, FIG. 3 is a graph which shows the relative density and thermal conductivity of a CNT/Cu metallic matrix material containing varying amounts of CNT as a volume fraction. See, J. Barcena, J. Maudes, J. Coleto and I. Obieta, “Novel Copper/Carbon Nanofibres Composites for High Thermal Conductivity Electronic Packaging,” incorporated herein by reference in its entirety. See also, A. Muhsan, F. Ahmad, N. Mohamed and M. Raza, “Nanoscale Dispersion of Carbon Nanotubes in Copper Matrix Nanocomposites for Thermal Management Applications,” Journal of Nanoengineering and Nanomanufacturing, Vol. 3, pp. 1-5, 2013, incorporated herein by reference in its entirety.

Most preferably, the thermally-conductive sleeve 112 comprises a CNT-enhanced copper nanomatrix material formed by spark-sintering a mixture of electrolytic copper powder and CNTs. Preferably, the copper powder is formed or processed in such a manner so as to form smooth-surfaced spheroidized particles having a size of about 3-8 μm. Powders comprising spherical or mostly spherical particles improves CNT dispersion and also improves the powder flow ability which is advantageous in subsequent molding and sintering processes.

Preferably the powdered mixture of electrolytic copper and 5-15 vol. % CNTs is subjected to a particles-compositing process which provides high inter-particle collision in a high-speed air flow. See, for example, K. Chu, H. Guo, C. Jia, F. Yin, X. Zhang, X. Liang and H. Chen, “Thermal Properties of Carbon Nanotube-Copper Composites for Thermal Management Applications,” Nanoscale Research Letters 2010, 5:868-874, incorporated herein by reference in its entirety. In this process, as further illustrated in FIG. 4, dendritic copper particles 151 are gradually deformed through mechanical impact into spheres 153 while agglomerated CNT ropes or lumps 155 are separated or disintegrated into individual CNTs 157 which then embed into the outer surface of each spherical copper particle 153 to form a composite CNT/Cu particle 159. As the process continues, the number of copper spheres and embedded CNTs increases and a composite powder with substantially uniformly dispersed CNTs is ultimately achieved. The resulting composite powder provides homogeneously dispersed CNTs where most of the CNTs are at least partially embedded and strongly attached to the copper powder rather than clinging weakly to the outer surface thereof.

In accordance with another embodiment of the invention, the annular cavity 108 (see FIG. 2) formed between the outer housing 107 and the motor 103 may be entirely or partially filled with a liquid cooling fluid (not show) such as dielectric oil having high electrical impedance. Advantageously, the cooling fluid can help cool the motor 103 by convectively conducting heat from the motor to the outer housing 107. In that case, the motor 103 may have either a sealed or vented housing depending on whether it is desired to directly expose the internal motor components to the cooling fluid. See, for example, U.S. Pat. No. 7,352,090 Guftafson, incorporated herein by reference in its entirety.

### Sealing and Leakage-Prevention

As noted above, a particularly difficult challenge with powered surgical instruments is ensuring long-term survivability and reliable operation through multiple surgical procedures and repeated cycles of autoclaving or sterilization. The autoclaving process creates a germ-hostile environment by exposing a device to high-temperature steam and alternating cycles of high and low pressure. Repeated exposure to this hostile environment can significantly shorten the useful life expectancy of powered surgical instruments having internal electric motors and other sensitive electronic components.

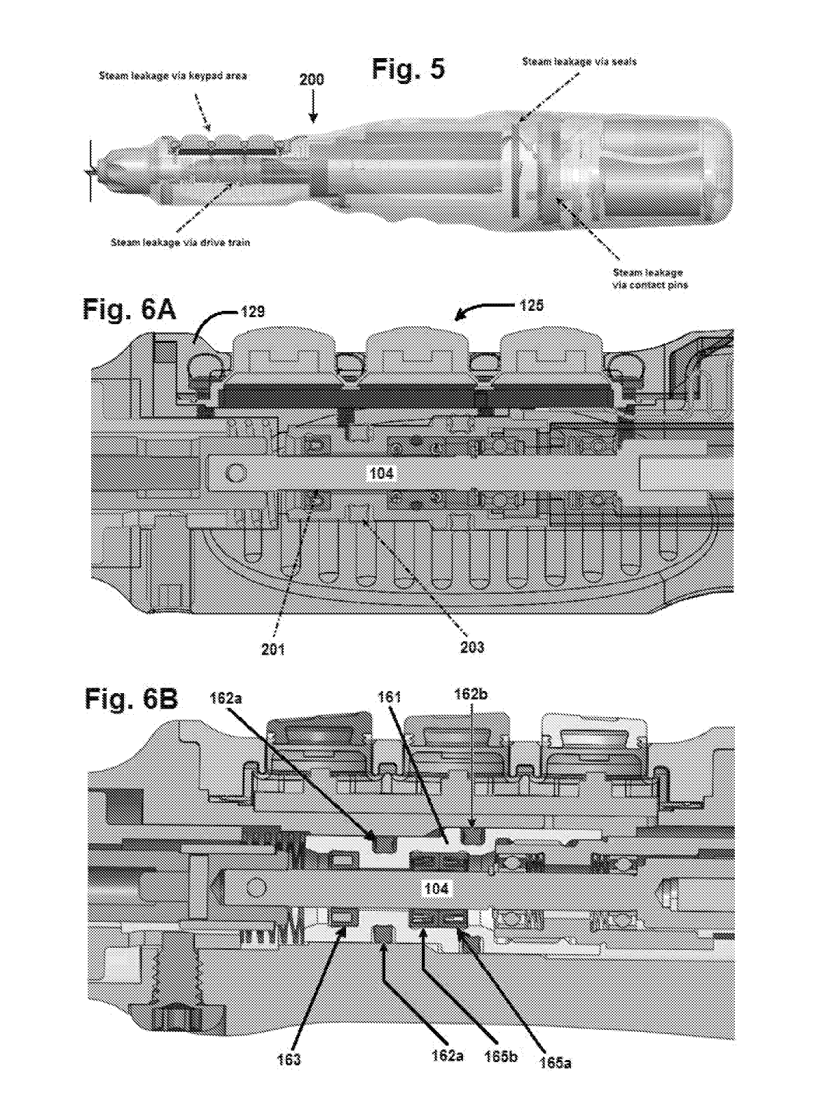

The primary cause of failure is ruptured, worn or damaged seals and concomitant damage caused to sensitive internal components from leakage of steam, fluids and/or other contaminants into the device. Another particular challenge is that leakage often cannot be detected until after significant permanent damage has already been done. FIGS. 5 and 6 illustrate major sources of potential leakage in a battery-operated motorized handpiece 200. These include: i) the drive train (e.g., lip seals 201, quad-O-ring seals 203), ii) the battery contact pins, iii) the endplate interface, and iv) the input/control keypad 125 and keypad housing interface 129.

### Drive Train

As illustrated in FIG. 6A, leakage around the driveshaft 104 can present particularly difficult challenges, especially in applications where saline solutions are used, such as in a shaver system. Lip seals 201, quad-O-rings 203 and other similar sealing systems are traditionally used to seal rotating or sliding mechanical components, such as drive shaft 104, against ingress of fluids or other contaminants. However, due to the sliding nature of the mechanical interface, these types of sealing systems are inherently prone to leakage as the seals and their mating mechanical components move, rotate, slide and eventually corrode, crack, pit, or wear down over time. As leakage paths eventually develop, saline solution and other fluid contaminants can be dragged or pulled underneath the seal by the moving mechanical component at the sealing interface. Once the seals have been breached, steam is then able to pass underneath the seals 201, 203 during the autoclave cycle and can get inside the motor and other sensitive components, causing irreparable damage and eventual failure of the device.

FIG. 6B is a partial-sectioned detailed schematic view of an improved drivetrain sealing system for a surgical handpiece having features and advantages in accordance with one embodiment of the present invention. The sealing system generally includes a seal housing 161 configured to interface with the fixed outer housing 107 and a movable drive shaft 104 extending through the seal housing 161. Two outer quad seals 161a, 161b are provided on stepped diameters to substantially prevent leakage between the outer housing 107 and the seal housing 161. A primary grease-filled lip seal 163 is provided between the seal housing 161 and the movable shaft 104 in order to seal the shaft 104 and substantially prevent ingress of steam during the positive phase of the autoclave cycle. A secondary lip seal 165a is provided between the seal housing 161 and the movable shaft 104 in order to seal the shaft 104 and substantially prevent ingress of steam during the negative phase of the autoclave cycle. Optionally, an additional lip seal 165b may be provided and positioned in an opposite direction from lip seal 165a to provide enhanced sealing against pressures in two different directions that occur during the pressurization and vacuum cycles of the autoclaving process. As a further design option, the lip seals 165a, 165b may be formed as a single one-piece bi-directional seal. The lip seals 163, 165a, 165b may be spring energized lip seals such as canted coil spring seal or u-channel seals positioned either on step-down diameters or on the same diameter. If desired, the lip seals 165a, 165b may be employed in further combination with an excluder seal (e.g., an annular seal with an x-shaped cross section, or a U-shaped finger type spring, or unidirectional canted coil seal).

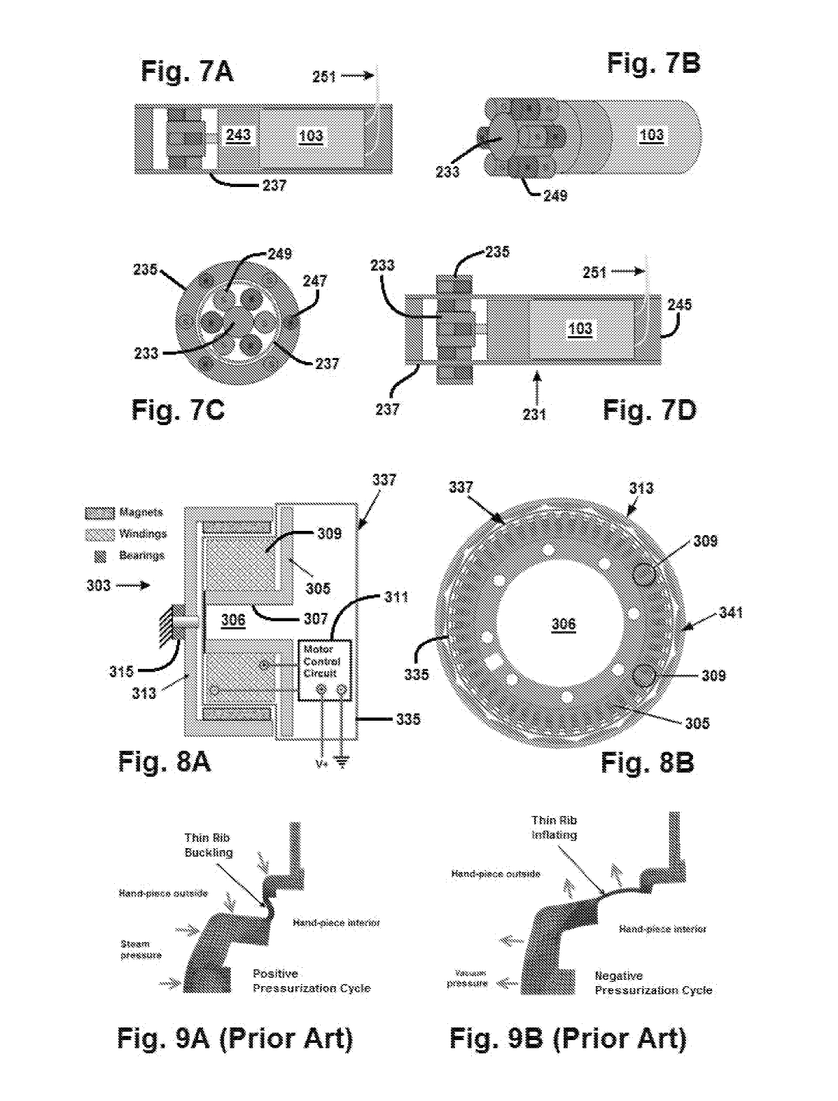

In another embodiment of a motorized handpiece having features and advantages of the present invention the drivetrain sealing system may be eliminated altogether (or at least eliminated as a potential source of leakage) by incorporating one or more motor or drive train elements configured to transmit torque or other mechanical forces through a wall of a sealed housing via a magnetic coupling (see, e.g., FIGS. 7A-D and 8A-B). A magnetic coupling is a commercially available coupling device, which connects motor and machine by permanent magnetic forces acting through the walls of a sealed vessel. They are typically used in closed systems for pumping sensitive, caustic, volatile, flammable, explosive or toxic solutions and in other similar applications where design requirements call for zero possibility of leakage or contamination. See, for example, published PCT application WO2013039144 to Hoshi, incorporated herein by reference in its entirety. Magnetic couplings are also commonly used in industrial-scale, deep-diving underwater rovers, submarines and the like where the pressure differential across a sealed containment vessel is just too great for a conventional lip-seal or quad-O-ring sealing system. See, for example, “Magnetically Coupled Drive,” posted by Eric Stackpole on Jun. 2, 2011 (http://openrov.com/forum/topics/_magnetically-coupled-drive, accessed 2013 Sep. 25).

Advantageously, magnetic couplers do not rely on lip seals or any other kind of sealing mechanism between moving parts because motion, torque, force and/or energy is transmitted directly through a fixed wall of a permanently sealed vessel or housing through magnetic force fields. Magnetic couplers also have the advantage of providing built-in torque limiting capability due to the fact that an applied torque in excess of the magnetic attraction forces between attracting magnetic components comprising the magnetic coupler will simply cause the magnetically-coupled components to slip passed one another. These are particularly desirable features in a motorized handpiece as disclosed and described herein.

In one embodiment the magnetic coupler 231 essentially comprises a miniaturized version of an industrial-grade magnetic coupler of the type commercially available, for example, from Dexing Magnet Tech Co., Ltd. As schematically illustrated in FIGS. 7A-D, the magnetic coupler 231 generally comprises an internal rotor 233 which is inserted into a containment vessel 237, and an outer rotor 235 which slips over the containment vessel 237. The containment vessel comprises a partially enclosed metal cylinder or can having an opening at one end. A sealing flange (not shown) may be provided and configured to secure and seal the containment vessel 237 to the wall of another containment structure (not shown), such as another sealed vessel. An optional backing plate (not shown) may also be provided and configured to mate with the sealing flange so as to form a tight compression seal that sandwiches, for example, the walls of a correspondingly configured sealed vessel or other structure, as desired. In order to provide a secure, leak-proof seal those skilled in the art will readily appreciate that one or more resilient seals or gaskets (not shown) may also be provided between the sealing flange, backing plate, and/or an intermediate support structure or component as desired.

The containment vessel 237 is preferably formed of a non-magnetic material having good mechanical strength, durability, and resistance to fatigue and corrosion. Medical-grade non-magnetic stainless steel, nickel, titanium and naval brass are preferred materials. Alternatively, other materials, including non-metals and metals having some magnetic properties, may be used with efficacy. The walls and overall design structure of the containment vessel 237 are configured to support a design pressure differential under full design-load conditions and maximum fatigue cycling without rupturing, cracking or leaking. The cylindrical walls of the containment vessel are preferably formed as thin as reasonably possible and as closely fitting to the internal rotor 233 as reasonably possible in order to minimize the gap between the internal rotor 233 and the containment vessel 237 when assembled together, as illustrated in FIG. 7A. Likewise, the external rotor 235 is preferably formed as closely fitting to the outer cylindrical walls of the containment vessel 237 as reasonably possible in order to minimize the gap between the external rotor 235 and the containment vessel 237 when assembled together, as illustrated in FIGS. 7C and 7D.

The inner and outer rotors 233, 235 are supported by one or more bearings (not shown) which are preferably arranged and supported in precision alignment with the containment vessel 237 and each other rotor 233, 235 so that the inner and outer rotors 233, 235 are free to rotate concentrically relative to one another and the fixed containment vessel along a common rotation axis. Multiple permanent magnets 249 are circumferentially arranged substantially flush with the outer surface of the inner rotor 233 and with substantially equal radial spacing. A corresponding number of permanent magnets 247 are arranged substantially flush with the inner surface of the outer rotor 233 and with corresponding radial spacing. These may comprise one or more rare-earth magnets such as, for example, nickel-plated neodymium magnets (aka NdFeB, NIB or Neo magnets). Neodymium magnets are permanent magnets made from an alloy of neodymium, iron and boron to form a Nd2Fe14B tetragonal crystalline structure and are one of the strongest types of permanent magnets commercially available. The maximum transmissible torque of the magnetic coupler 231 is determined by the number, size and type of permanent magnets incorporated into the device and the size of the gap between the internal and external rotors.

A brushless DC motor 103, optional motor control circuitry (not shown), and an optional gearbox 243 are all mounted within the sealed containment structure 237. Electrical leads 251 bring electrical power from an external power source (e.g., a battery) to power the motor 103. The output shaft of the motor 103 is mechanically coupled to the internal rotor 233. The ends of the containment structure 237 are preferably permanently sealed with a potting material, a sealing compound, sealing plate or other sealing device as desired. Alternatively, one or more ends of the containment structure 237 may be removably sealed with a removable plate, cover or the like in order to provide, for example, repair and/or maintenance access to the sealed cavity within.

As described above, the internal and external rotors 233, 235 are each mounted independently on separate bearings that are fixed relative to the cover 237 such that each rotor spins freely around a common spin axis. A first set of magnets 247 are mounted inside a ring of material (forming the external rotor 235) having an inner diameter at least slightly larger than the outer diameter of the cylindrical isolating cover 237. A second set of magnets 249 are mounted on a cylindrical hub (forming the internal rotor 233) having a maximum outer diameter at least slightly smaller than the internal diameter of the isolating cover 237. For example, FIGS. 2C and 2D show the front and side cross sections of the outer ring with magnets 247 surrounding corresponding inner magnets 249. While only the outer ring 235 is shown for illustrative purposes, those skilled in the art will readily appreciate that the outer ring may also be mechanically coupled to an output driveshaft 239 (not shown). Referring again to FIGS. 7C and 7D, magnets 247, 249 are preferably configured in alternating polarity in order to provide maximum torque transfer due to oppositely polarized magnets strongly attracting each other while strongly repelling neighboring magnets.

In operation, a brushless DC-powered motor 103 provides torque to the internal rotor through an optional gearbox 243. This drives the internal rotor 233 at the same speed and in the same direction as the motor 103. This rotational motion and torque is transferred to the external rotor 235 (up to a design threshold torque limit) due to the arrangement of oppositely polarized magnets 247, 249 strongly attracting each other while strongly repelling neighboring magnets. The resulting rotational output and torque can be mechanically coupled to other drivetrain components in any number of conventional and well-known ways to power other rotating and/or reciprocating elements as required or desired. Alternatively, those skilled in the art will readily appreciate that alternative rotor embodiments and other magnetically coupled structures may also be used to achieve similar advantages as taught herein, including, without limitation, flat rotating disks, and linearly sliding or reciprocating plates, rods, tubes and the like.

In another embodiment, as illustrated in FIGS. 8A and 8B, the motor 103 (e.g., from FIG. 2) may comprise a modified motor 303 comprising, for example, a brushless DC motor of the type having an external rotor that magnetically communicates torque with a fixed stator through the walls of a sealed vessel. Like the magnetic coupler device 231 described above in connection with FIGS. 7A-7D, a motorized handpiece or other surgical instrument incorporating such a modified motor 303 would have no critical need rely on lip seals or any other kind of sealing device between movable drivetrain components because motion, torque, force and/or energy is transmitted through a fixed wall of an enclosed sealed housing via forces of magnetic attraction and/or repulsion.

As schematically illustrated in FIGS. 8A and 8B the modified motor 303 generally comprises: i) a fixed inner stator 305 enveloped within and conforming to the cylindrical walls 335 of a sealed containment vessel 337 or other containment system, and ii) an external rotor 313 concentrically arranged outside of the cylindrical walls 335 of the containment vessel 337 and supported by one or more precision bearings 315 so as to rotate about the inner stator 305, as illustrated. Advantageously, and as will be readily recognized and appreciated by those skilled in the art, the external rotor 313 may be formed as a purely mechanical component (e.g., comprising permanent magnets supported by a simple ring structure) such that it is not easily susceptible to damage or failure caused by exposure to moisture, steam, debris, saline solution and/or other contaminants. On the other hand, the electrically-powered inner stator 305 and associated motor control circuitry 311 are all preferably safely contained within a robustly sealed containment vessel 337 or other containment system, as desired.

The containment vessel 337 is preferably formed of a non-magnetic material having good mechanical strength, durability, and resistance to fatigue and corrosion. Medical-grade non-magnetic stainless steel, nickel, titanium and naval brass are preferred materials. Alternatively, other materials, including non-metals and metals having magnetic properties, may be used with efficacy. The walls and overall design structure of the containment vessel 337 (shown here schematically and without intending any specific structural limitations or requirements) are configured to support a design pressure differential under full design-load conditions and maximum fatigue cycling without rupturing, cracking or leaking. The cylindrical walls of the containment vessel are preferably formed as thin as reasonably possible and as closely fitting to the internal stator 305 as reasonably possible in order to minimize the magnetic gap between the internal stator 305 and the external rotor 313, as illustrated in FIGS. 8A and 8B. In one embodiment, the cylindrical walls 335 are sized and shaped to closely conform to the stator 305 such that they are abutting or nearly-abutting against the outer surface of the inner stator 305, as illustrated. For example, a suitably-formed cylindrical containment vessel 337 could be assembled over the inner stator by slip-fitting or press-fitting. Alternatively, or in addition, a coating, sealant, adhesive or other suitable surface sealing material (not shown) may be directly applied to and/or formed with the outer surface of the inner stator 305 in order to provide robust protection against potentially-damaging moisture, steam and other contaminants.

In one embodiment, internal stator 305 comprises a metallic core 307 formed from, for example, stacked plates of laminated steel or other ferromagnetic materials (e.g., various alloys of iron, nickel, cobalt and manganese). The stacked plates are configured to carry two or more windings 309 arranged in a star pattern (Y), delta pattern (Δ), or other pattern as desired or expedient. The Y pattern gives high torque at low RPM while the A pattern gives low torque at low RPM. Each stator winding 309 is configured to produce a corresponding magnetic field or magnetic pole when energized by an electric current. The stator 305 may comprise any number of windings 309 and corresponding magnetic poles, as desired or expedient, although at least two or more is preferred. Increasing the number of poles provides better torque performance (e.g., higher torque and more precise torque control) but at the cost of reducing the maximum possible speed. More preferably, the stator 305 comprises at least 16 or more windings 309 and corresponding magnetic poles.

The laminated plates forming the core 307 of stator 305 can be slotted or slotless, as desired or expedient. A slotless core has lower inductance and, thus, can run at higher speeds. Alternatively, a slotted core (see, e.g., FIG. 4B) may be used in order to reduce costs (less windings required for a given torque) or where the design speed of the motor 303 is relatively low. In one embodiment, the core 307 of stator 305 further comprises or defines an internal cavity 306. Optionally, this cavity 306 may be entirely or partially filled with a liquid cooling fluid (not show) such as dielectric oil having high electrical impedance. Such structures and features can be provided, for example, to help cool the motor 303 by convectively conducting heat away from the stator 305 to the walls 335 of the outer enclosure 337.

As noted above, an external rotor 313 is provided and is concentrically arranged outside of the cylindrical containment walls 335 and supported by one or more bearings 315. Preferably the bearings 315 support the external rotor 313 in precision alignment with the containment vessel 337 such that the external rotor 313 is free to rotate relative to the containment vessel 337 about its central axis. The external rotor 313 may be similar in overall design and construction to the external rotor 235 illustrated and described above in connection with FIGS. 7A-7D. Multiple permanent magnets 341 (preferably corresponding to the number of magnetic poles of stator 305) are circumferentially arranged and preferably equally radially spaced along an inner surface of the external rotor 313. These may comprise one or more rare-earth magnets such as, for example, nickel-plated neodymium magnets. For a given energizing current applied to stator 305 the maximum transmissible torque of the motor 303 will be determined by the number, size and type of permanent magnets 341 incorporated into the external rotor 313 and the size of the gap between the internal stator 305 and the external rotor 313. If desired, a coating, sealant, adhesive or other surface sealing material (not shown) may be directly applied to or formed with the outer surface of the external rotor 313 in order to provide robust protection against potentially-damaging moisture, steam and/or other contaminants. Alternatively, those skilled in the art will readily appreciate that alternative rotor and stator embodiments and other magnetically coupled structures may also be used to achieve similar advantages as taught herein, including, without limitation, rotors or stators comprising flat opposing disks, linear motors and actuators, sliding or reciprocating plates, rods, tubes and the like.

Operation of the modified motor 303 follows the same basic underlying principles that apply to typical brushless DC motors. Motor control circuit 311 selectively applies a voltage from an external voltage source across one or more of the stator windings 309 which, in turn, causes a current to flow creating one or more corresponding magnetic fields. Each magnetic field attracts and/or repels one or more nearby permanent magnets 341 that form part of the external rotor 313. This imparts torque on the rotor 313 and causes it to rotate either clockwise or counterclockwise, as desired. As the rotor 313 rotates relative to the stator 305, an internal sensor (e.g., a Hall effect sensor, or optical encoder, not shown) senses the angular position of the rotor 313 relative to the stator and provides a corresponding control feedback signal to motor control circuit 311. The motor control circuit 311 uses the control feedback signal to selectively apply voltage and current in to the stator windings in a predetermined pattern and in alternating polarity so as to create multiple commutating magnetic fields that drive the rotor 313 in a desired direction and with a desired speed and torque. Preferably, the stator 305, windings 309, and all associated motor control circuitry 311 are safely sealed within the housing 337.

### Contact Pins

In a traditional battery-operated handpiece the contact pins that provide connection to the batteries in the back of the hand-piece are typically potted for sealing. However, depending on environmental conditions (e.g., such as the moisture content in the air at the time of potting), this type sealing system may not always function reliably to prevent ingress of moisture, steam and other contaminants in the harsh environment of the autoclaving process. The problem is further compounded by the fact that there is typically a small gap between the contact pin and the handpiece housing which provides a leakage path. As the handpiece goes through multiple autoclave cycles, the potting tends to lose its sealing capacity due to steam leaking past the gap between the housing and the contact pin.

Accordingly, in one embodiment of the present invention, as illustrated in FIGS. 14A and 14B, dual quad-O-ring seals 353 are provided around each contact pin 349 at stepped diameters from a smaller diameter (inner seal) to a larger diameter (outer seal). If desired, additional sealing pressure may be applied to the stepped quad-O-ring seals 353 via a C-clamp 355, as illustrated in FIG. 14B. Advantageously, the C-clamp 355 substantially prevents the contact pin from moving away from the housing during the negative cycle of the autoclaving process.

### Input Keypad

Keypads in current-generation hand-piece designs are particularly prone to leakage and failure. This is because most conventional keypads are not well-designed for the autoclave cycle. As illustrated in more detail in FIGS. 9A and 9B, conventional keypads typically comprise flexible silicone-molded buttons supported by thin elastic ribs or webbing, which provide a desired spring-back response for normal keypad operation. However during multiple vacuum and positive pressurization cycles, the thin rib often presents a point of mechanical fatigue, cracking or rupturing and therefore can become a leakage point. In particular, during each positive pressurization cycle a conventionally-designed keypad buckles down as shown in FIG. 9A. Then during each vacuum cycle, air inside of the handpiece expands causing the keypad buttons to inflate like a balloon and lift up. This in turn causes the connecting rib to also inflate, bulge out and stretch as shown in FIG. 9B. Repeated cycles of buckling, inflating, stretching and relaxing eventually causes the rib to fail and leak.

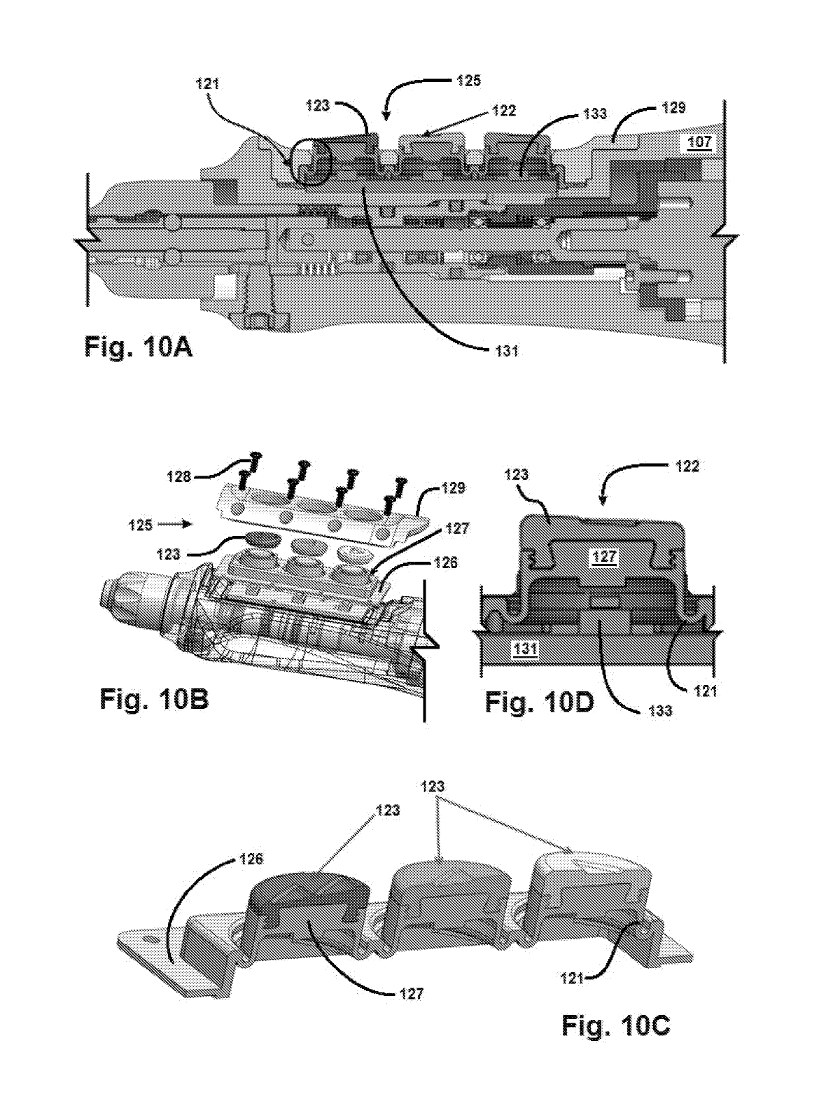

FIGS. 10A and 10B illustrate one embodiment of an improved keypad design for a surgical handpiece having features and advantages of the invention. As illustrated in FIG. 10A, the improved keypad 125 generally comprises an integrally molded silicone upper portion 127 comprising one or more (as illustrated, three) flexibly suspended depressible buttons 122. Below the upper portion 127 a lower portion 131 is provided comprising a printed circuit board (PCB) or other electrically-conductive support structure. As illustrated, each depressible button 122 is positioned above a corresponding micro-switch 133 or other switch-closure element configured to sense or electrically communicate when each corresponding button 122 is depressed. A cover plate 129 comprising a comparatively rigid frame entraps each button 122 within a defined limited range of motion and secures and seals the entire keypad assembly 125 to the outer housing 107.

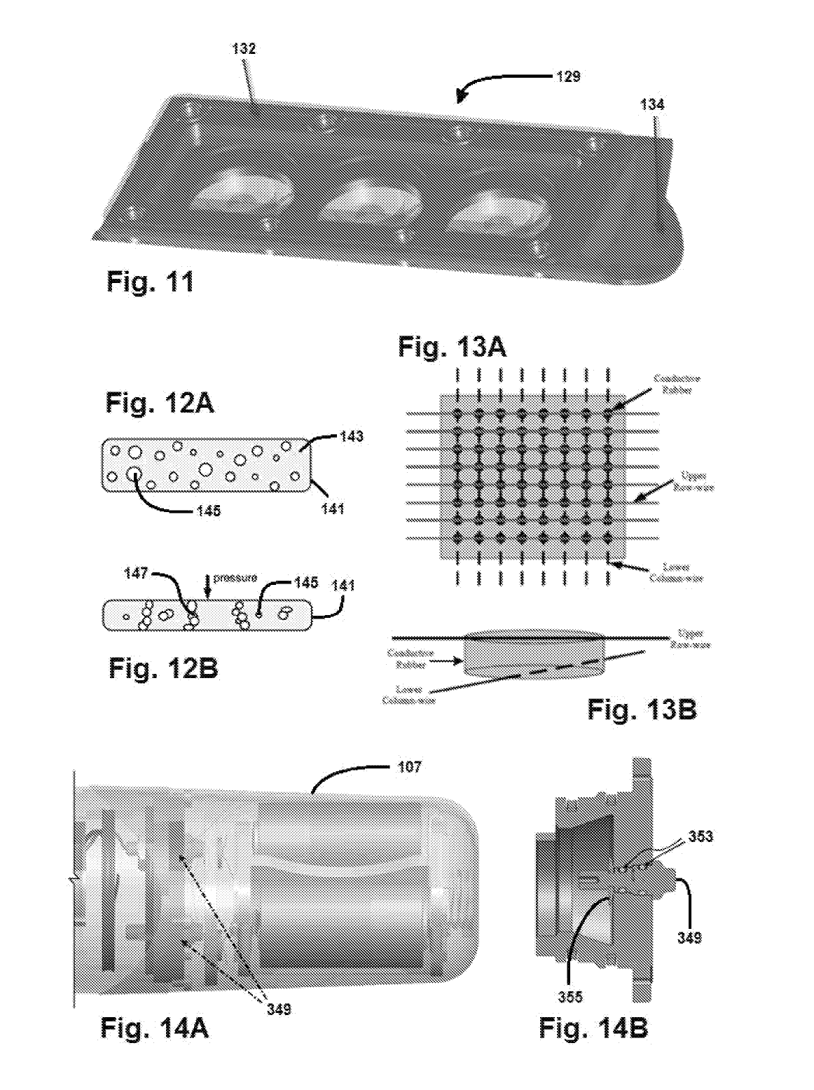

As illustrated in FIG. 10B, the silicone upper portion 127 preferably includes an outer flange portion 126 which, when compressed between the housing 107 and the cover plate 129, provides a gasket-like seal. Screws 128 hold the cover plate 129 to the housing and are preferably evenly spaced around the periphery of the cover plate and sufficiently tightened so as to provide adequate sealing of the cover plate 129 against the housing. Optionally, the screws 128 may be coated with DLC and/or other lubricious coatings in order to reduce the coefficient of friction of the screws, increase screw preload for a given torque and reduce screw loosening during multiple thermal cycles. FIG. 11 is a bottom plan view of a preferred keypad cover plate 129 design having features and advantages in accordance with the present invention. The portion of the cover plate 129 that engages outer flange portion 126 preferably includes a small channel 132 to provide improved sealing engagement with the flange portion 126. An optional shoulder 134 may also be provided in order to precisely limit the travel of the cover plate 129 when it is seated against the housing and tightened by the screws 128.

As illustrated in more detail in FIGS. 10C and 10D, preferably each button 122 is flexibly suspended by a relatively thick elastic rib 121 (preferably thicker than 0.44 mm) having a generally U-shaped lower portion. The relatively thick U-shaped rib supporting each button 122 advantageously accommodates flexing and bending with significantly less stress and fatigue than conventional designs. Each button 122 also preferably includes a sloped cap 123 formed of a medical-grade metal (e.g., stainless steel) or other relatively rigid material. The metal cap 123 provides mechanical rigidity across the upper surface of each button 122 to provide durability and increased integrity. The metal cap 123 also helps resists buckling and inflating of the button 122 and the connecting ribs 121 during multiple autoclaving cycles. Preferably the metal caps 123 are integrally molded with the upper portion 127 of the keypad 125 using an insert molding process or other suitable molding process as those skilled in the art will readily appreciate.

Alternatively and/or in addition, the keypad 125 may be formed partially or entirely from pressure-sensitive conductive rubber. For example, the entire upper keypad portion 127 may be formed of pressure-sensitive conductive rubber. Alternatively, each micro-switch 128 an/or the entire lower keypad portion 131 may comprise one or more sensing elements formed of pressure-sensitive conductive rubber configured to sense a force or pressure exerted on one of more of the input buttons 122.

Pressure-sensitive conductive rubber can comprise virtually any kind of conductive polymer that is configured to electrically respond to pressure. As illustrated in more detail in FIGS. 12A and 12B, pressure-sensitive conductive rubber 141 is typically formed from a base material 143 comprising a non-conductive resilient polymer, such as silicone or polyurethane, to which is added one or more conductive fillers 145, such as carbon black, silver, graphite, CNTs, or the like. The resulting composite material may be formed with a wide variety of conductivity properties and/or other electrical properties as desired, by varying the type(s) and amount(s) of conductive filler(s) added to the polymer matrix. For example, the electrical conductivity of carbon black is far larger than the conductivity of silicon rubber and so different mixtures or composites containing different ratios of carbon and silicon will have different electrical conductivity properties.

As illustrated in FIGS. 12A and 12B, the rubber's pressure-sensitive property derives from mechanical deformation in reaction to an applied pressure. When little or no pressure is applied, the conductive particles 145 within the insulating polymer matrix 143 are positioned relatively far apart from each other (as illustrated in FIG. 11A), such that the overall resistance to electrical current flow through the composite polymer 141 is relatively large. However, when sufficient pressure is applied, the material deforms such that the conductive particles 145 are forced closer together and/or form chains of contact 147 (as illustrated in FIG. 12B) providing additional conductive paths through the polymer matrix. This in turn reduces the overall resistance to electrical current flow through the composite polymer 141. This change in resistivity can readily be detected by an appropriately configured electrical circuit, as persons skilled in the art will readily appreciate. For example, in the case of a push-button or other user-input element formed from or acting upon a pressure sensitive conductive rubber, a threshold change in resistivity can readily be detected and communicated to an input/control system for thereby providing user control of an associated device.

In another alternative embodiment, the keypad 125 may be formed partially or entirely from a compressible dielectric material sandwiched between one or more conductive plates. Each button or other input element may be formed from two or more layers of silicone rubber, coated with one or more parallel lines or sheets of conductive material (e.g., carbon black, graphite, CNTs) glued together to form a pressure-sensitive variable capacitor, as illustrated in FIGS. 13A and 13B. When sufficient pressure is applied the compressible dielectric compresses, forcing the conductive lines or plates closer together, thereby changing the electrical capacitance of the pressure-sensitive variable capacitor. This change in capacitance can readily be detected by an appropriately configured electrical circuit, as persons skilled in the art will readily appreciate. For example, in the case of a push-button or other user-input element formed from or acting upon a pressure-sensitive variable capacitor, a threshold change in capacitance can be readily detected and communicated to an input/control system for thereby providing user control of an associated device. See, for example, U.S. Patent Application 2007-0257821 to Son, incorporated herein by reference in its entirety.

In another alternative embodiment, the keypad 125 may include one or more touch sensor elements or other solid-state electronic switches (not shown) activated by human touching and/or pressing of a finger. For example, these may include one or more solid state touch switches of the type wherein a user's finger (either bare or covered with a latex glove, for example) touching or pressing down upon one or more electrically conductive elements causes a detectible change in one or more electrical properties of the electrically conductive element (e.g., resistance or capacitance) causing a corresponding state change in an electrically coupled solid state electronic switching device, such as a MOSFET or PNP transistor. See, for example, U.S. Pat. No. 4,063,111 to Dobler, the entire contents of which is incorporated herein by reference.

In another alternative embodiment, the keypad 125 may comprise one or more solid-state piezoelectric input devices (not shown) such as the type sold by Burns Controls Company (e.g., Stainless Steel Piezo Switch, 22 mm, Blue LED Ring Illuminated, Product Code 07225198). Piezo switches are solid-state devices that directly convert mechanical stress (e.g., pressing down of a finger) into an electrical signal (e.g., a voltage or current). With no electrical switch contacts or moving parts they are extremely durable and reliable even in the harshest of environments.

Piezoelectricity refers to a unique property of certain materials such as quartz, Rochelle salt, and certain solid-solution ceramic materials such as lead zirconate-titanate (Pb(Zrl-xTix)03) (“PZT”) that causes induced stresses to produce an electric voltage or, conversely, that causes applied voltages to produce an induced stress. In a “generator” mode, electricity is developed when a piezoelectric (“piezo”) crystal is mechanically stressed. Conversely, in a “motor” mode, the piezo crystal reacts mechanically when an electric field is applied.

PZT is one of the leading piezoelectric materials used today. It can be fabricated in bimorph or unimorph structures (piezo elements), and operated in flexure mode. These structures have the ability to generate high electrical output from a source of low mechanical impedance or, conversely, to develop large displacement at low levels of electrical excitation. Typical applications include force transducers, spark pumps for cigarette lighters and boiler ignition, microphone heads, stereophonic pick-ups, etc.

### Motion Sensors

Alternatively and/or in addition to the input keypad 125, those skilled in the art will readily appreciate that one or more motion sensors may also be provided for sensing various motion- or gesture-based user input signals. See, for example, U.S. Pat. No. 8,286,723 to Puzio. In one preferred embodiment, sensed motions or gestures may include, for example and without limitation: i) full-body translational or rotational movement of the handpiece 100, ii) absolute or relative position, angle, or orientation of the handpiece in free space, iii) amount of input torque and/or force applied by a user to the handpiece, iv) amount of reaction torque and/or force applied by a user in resistance to translational or rotational movements of the handpiece, or v) tapping, jerking, shaking, or dropping the handpiece.

Silicon microchip input sensors comprising micro-electro-mechanical systems (MEMS) or nano-electro-mechanical systems (NEMS) devices are particularly preferred due to their small size (less than 1 cubic cm), light weight and (at least in the case of MEMS-based devices) their wide commercial availability and moderate cost. For example, a wide variety of MEMS-based accelerometers, gyros, proximity sensors, and geomagnetic sensors are commercially available for a variety of applications ranging from consumer gaming applications and smartphones to sophisticated missile guidance systems. MEMS sensors generally comprise two components: i) a mechanical sensing element that detects a motion, force or other physical condition desired to be sensed, and ii) an application-specific integrated circuit (ASIC), that amplifies and transforms the response of the mechanical sensing element into an electrical signal.

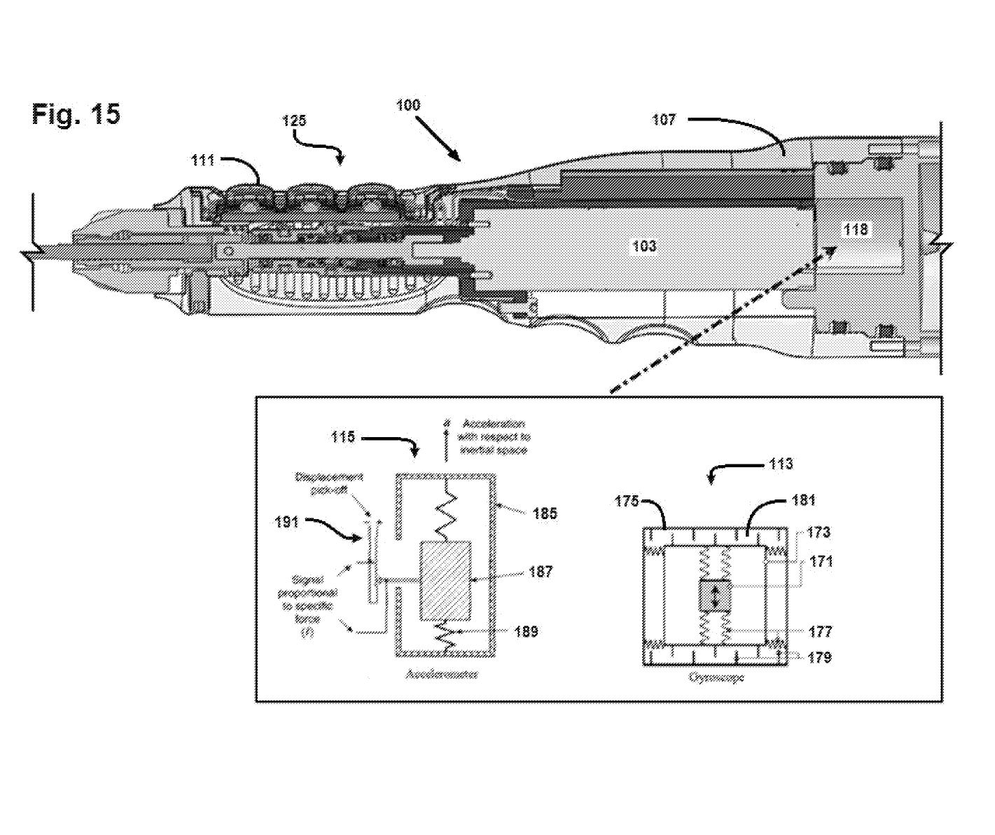

As schematically illustrated in FIG. 15, a typical silicon microchip MEMS gyroscope 113 generally comprises an inner frame 173 and an outer frame 175 resiliently supported relative to one another and having a small gap 181 configured to accommodate small amounts of relative movement. A vibrating mechanical element (illustrated schematically as a proof mass 171) is provided within the inner frame 173 and is supported by one or more resilient supporting elements (illustrated schematically as springs 177). In operation the proof mass 171 is caused to vibrate at a desired frequency. When the outer frame 175 of the gyro 113 is then rotated (e.g., by one or more motions imparted on the surgical handpiece 100), the vibrating mechanical element 171 experiences Coriolis acceleration. This in turn causes relative movement between the inner and outer housings 173 and 175 which is sensed as a change in the electrical capacitance between adjacent conductive sensing plates 179.

In one embodiment, a silicon microchip MEMS gyro is selected comprising an ADXRS80 integrated microchip available from Analog Devices. This gyro is capable of providing accurate angular displacement measurements in exceedingly harsh environments with temperatures ranging from −40° C. to 125° C. The ADXRS80 gyro is also relatively efficient, consuming only 6 milliamps under typical conditions, and has a relatively small overall envelope dimension of (˜10 mm×10 mm) in the 16-lead SOIC Cavity Package. Where greater accuracy and precision is required, a more-expensive tactical- or inertial-grade MEMS or NEMS gyro may be used instead of and/or in addition to a lower-grade MEMS or NEMS gyro. See, for example, A. Sharaf, “A Fully Symmetric and Completely Decoupled MEMS-SOI Gyroscope, Sensors & Transducers” (Apr. 1, 2011), incorporated herein by reference in its entirety. Alternatively, multiple lower-grade MEMS or NEMS gyro sensors may be used in order to provide multiple redundancy and/or failure recovery in the event one or more gyro sensors should fail to provide accurate readings.

As schematically illustrated in FIG. 15, a typical silicon microchip MEMS accelerometer 115 generally comprises a case 185 in which an inertial mass (illustrated schematically as a proof mass 187) is resiliently supported by one or more resilient supporting elements (illustrated schematically as springs 189). The case 185 is configured to accommodate small amounts of relative linear movement of the proof mass 187 along an axis defining a sensing axis of the accelerometer 115. Under steady state conditions, gravitational acceleration forces acting downward on the mass 187 reach equilibrium with countering forces exerted by springs 189. However, when the case 185 is subjected to an acceleration along its sensing axis (e.g., by one or more motions imparted on the surgical handpiece 100), the inertial mass 187 tends to resist the change in movement. This causes the inertial mass 187 to be displaced with respect to the outer case 185. The amount of displacement (and therefore the amount of acceleration) can be electrically measured by, for example, a linear potentiometer 191 and/or a variable capacitor device (not shown) formed by one or more movable and static conductive plates.

In one embodiment, a silicon microchip MEMS accelerometer is selected comprising an ADXL312 integrated microchip available from Analog Devices. The ADXL312 is a small, thin, low power, 3-axis accelerometer contained on a single, monolithic IC. It provides high resolution (13-bit) measurement up to ±12 g with digital output data formatted as 16-bit twos complement accessible through either a SPI (3- or 4-wire) or 120 digital interface. The device is capable of providing accurate acceleration measurements in exceedingly harsh environments with temperatures ranging from −40° C. to 125° C. The ADXL312 accelerometer is also highly efficient, consuming only 57 μA in measurement mode and 0.1 μA in standby mode, and has a relatively small overall envelope dimension of (˜5 mm×5 mm). Where greater accuracy and precision is required, a more-expensive tactical- or inertial-grade MEMS or NEMS accelerometer may be used instead of and/or in addition to a lower-grade MEMS or NEMS device. Alternatively, multiple lower-grade MEMS or NEMS accelerometers may be used in order to provide multiple redundancy and/or failure recovery in the event one or more accelerometers should fail to provide accurate sensor readings.

While MEMS-based or NEMS-based gyros, accelerometers and geomagnetic sensors are specifically disclosed and described herein as suitable input sensor devices for use in accordance with one or more embodiments of the present invention, those skilled in the art will readily appreciate that a wide variety of other sensing devices may be used instead of and/or in addition to those specifically disclosed. Other suitable motion sensor devices include, for example and without limitation, non-MEMS-based sensors, tilt sensors, inertial sensors, shock or impact sensors, drop sensors, vibration sensors, proximity sensors, touch or grip sensors, and the like. Any one or more of these may be provided within an internally-sealed cavity 118 and packaged therein so as to mechanically-isolate the sensors from any large undesired shocks and vibrations such as caused by dropping or striking the handpiece 100 against a hard surface. See, for example, FIGS. 23A and 23B and the associated disclosure contained herein.

### Condition Sensors

In addition to the various motion- and position-based sensors disclosed and described above, the present invention also specifically contemplates the use of one or more condition sensors for sensing various operating and non-operating (e.g., during autoclaving) conditions relevant to the handpiece 100, such as heat, temperature, pressure, moisture, humidity, leakage, and the like. Sensor data from these and/or any other sensors may be used for purposes of providing improved feedback control and/or for purposes of providing improved performance and maintenance monitoring.

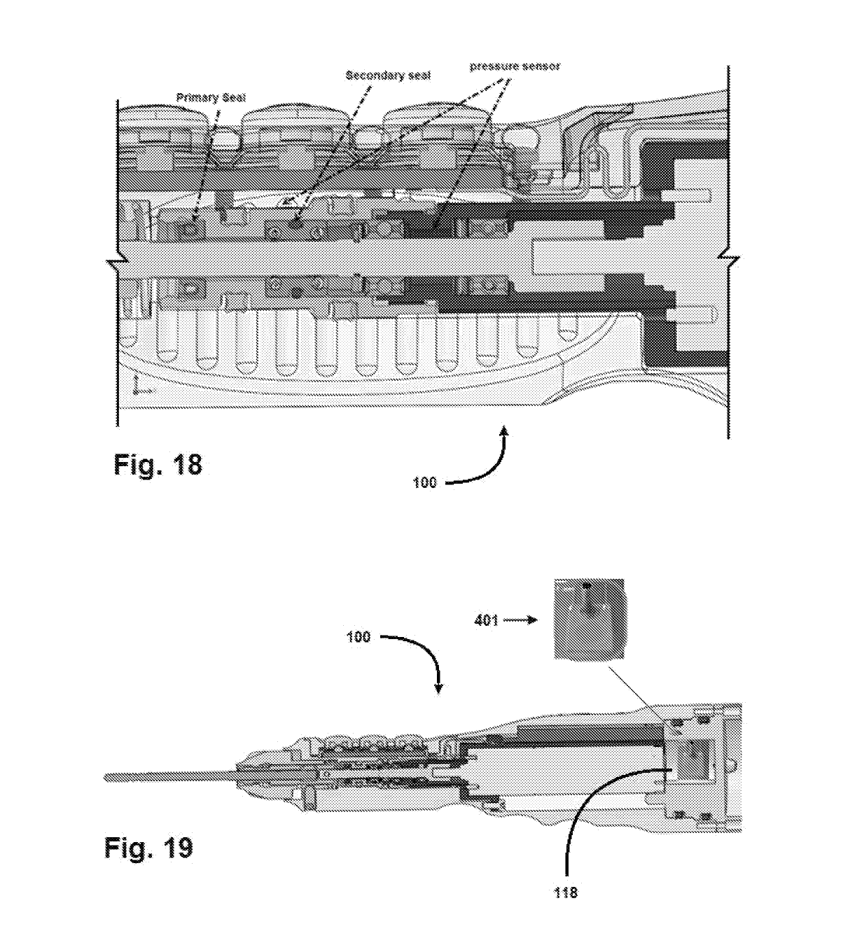

In accordance with one embodiment, sensor data may be used to provide early failure detection and/or recommended testing, inspection or maintenance of a motorized handpiece 100. For example, when the lip seals around the driveshaft begin to fail (e.g., see FIG. 6), the very first indication would be a change in the pressure and humidity inside the hand-piece during and after the autoclave cycle. As illustrated schematically in FIG. 18, a combination of humidity and/or pressure sensors can be provide between the primary and secondary seals such that an internal computerized monitoring system (discussed in more detail below) can monitor and detect when steam or saline has potentially breached the seals and provide maintenance recommendations to a user based thereon. The internal computerized monitoring system can also automatically preferably shut down operation of the handpiece or prevent starting or further operation of the handpiece following an autoclave cycle where significant leakage is been detected, thus preventing costly and potentially irreparable damage.

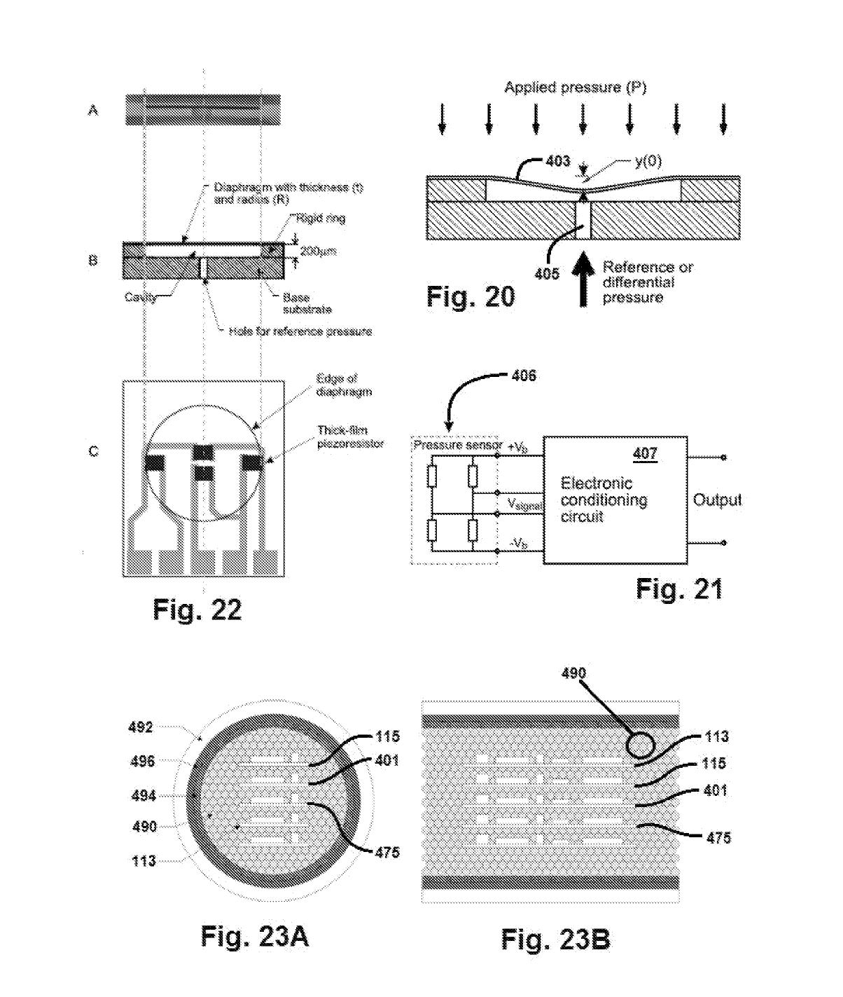

In another embodiment, as illustrated schematically in FIG. 19, a pressure sensor 401 is provided and disposed within an internal cavity 118 provided within the handpiece 100. In one embodiment the pressure sensor 401 may comprise a pressure-sensing membrane 403 that flexes in response to a sensed pressure differential across the membrane, as illustrated in FIGS. 20-22. For example, the membrane 403 may be exposed on one side to ambient air outside of the housing (e.g., through a small orifice 405 formed in a supporting base structure 407 and/or through a wall of the outer housing 107) and on other side to air or fluid inside the sealed housing 107. Those skilled in the art will readily appreciate that movement of the membrane 403 can be detected, for example, by a suitably configured electronic circuit configured to detect changes in the capacitance, resistance, piezo-resistance or piezoelectric properties of the membrane 403. For example, FIG. 21 illustrates a one embodiment of a detection and signal conditioning circuit 406 comprising a simple Wheatstone bridge 407.

In another embodiment a MEMS-based pressure sensor is utilized instead of or in addition to the pressure sensors described above. MEMS based sensors are mechanically similar to conventional pressure sensors. The main difference is that MEMS/micro sensors are made using a silicon and/or a silicon nitride diaphragm. Silicon nitride has fracture stress of 2200 GPa (Si 170 GPa) and an ultimate strain of 7.8×10⁻³ (Si 0.7×10⁻³). Silicon and silicon nitride based pressure sensors use micro mechanical structures i.e. cantilevers, plates or diaphragm etc. for pressure measurement at micro scales which offers several advantages, such as small size, high sensitivity, wide dynamic range, high stability, and easy integration with CMOS electronics. Typical MEMS pressure sensors include a diaphragm and piezoresistors made from silicon and/or silicon nitride. The diaphragm is typically made by anisotropic etching at the back side of the bulk silicon whereas other sensing element i.e. piezoresistors are embedded into the diaphragm. When pressure is applied, the diaphragm generates a mechanical signal which in turn can be converted into an electrical signal by a suitable electrical circuit.

Silicon nitride is a preferred diaphragm material for a MEMS based pressure sensor. Silicon nitride has a high strength (e.g., yield strength of 14 GPa), which can withstand the maximum load without breaking the diaphragm. At the same time, higher mechanical sensitivity, which is governed by the mechanical dimension of the diaphragm, can be achieved by reducing the diaphragm size. This material has low value of CTE as well as low thermal conductivity. Silicon nitride has a dielectric constant of 6.7, which remains pretty constant over the 10-60 GHz frequency range.

This material has very high resistively greater than 1012Ω from room temperature to 200° C., and thus can be used as an insulation layer in MEMS switches. It has demonstrated a tensile strength greater than 25,000 psi at elevated service temperatures and excellent thermal shock resistance. Silicon nitride experiences very little volume change, thereby making it most desirable for a MEMS assembly where close dimensional tolerances are very critical for instance in autoclave application.

To convert mechanical stress generated in diaphragm due to external load, into an electrical signal, monocrystalline silicon resistors may be used as the sensing elements which offers significant advantages i.e. high piezoresistive coefficient, low hysteresis, long term stability etc. One or more such resistors can be used, which directly experience the stress from the diaphragm, and convert mechanical strain into electrically-measurable resistance. Resistors may be oriented parallel to the diaphragm edges and/or perpendicular to the edges in order to sense the applied pressure. The resistors may be arranged in Wheatstone bridge configuration as those skilled in the art will readily appreciate.

Another type of MEMS based pressure sensor are those made from various types of ceramic materials. These can provide a very useful alternative to silicon-based pressure sensors, especially in harsh environments and at high temperatures. The laminated 3D structures made using low-temperature co-fired ceramic (LTCC) are especially practical for so-called ceramic MEMS. Silicon pressure sensors currently dominate the market, but in some demanding applications thick-film technology and ceramic materials can be used for the fabrication of sensor systems, i.e., ceramic or thick-film pressure sensors. In comparison with semiconductor sensors they are larger, more robust and have a lower sensitivity, but they have a high resistance to harsh environments.

LTCC technology and materials are suitable for making the ceramic structure of a thick-film pressure sensor, which can work in a wide temperature range and in different media (gasses, liquids)—in this case when the steam leaks inside the handpiece. This structure consists of a circular, edge-clamped, deformable diaphragm that is bonded to a rigid ring and the base substrate. In the base substrate is the hole for the applied reference or differential pressure. These elements form the cavity of the pressure sensor. The depth of the cavity is large enough to accommodate maximum design flexure and depends on the thickness of the rigid ring. A typical LTCC pressure sensor can measure pressures in the range from 0 to 100 kPa, and have a typical burst pressure of about 400 kPa.

In another embodiment a piezoresistive ceramic pressure sensor is utilized instead of or in addition to one or more of the pressure sensors described above. A piezoresistive ceramic pressure sensor is based on the piezoresistive properties of thick-film resistors that are screen-printed and fired onto a deformable ceramic diaphragm. The piezoresistive ceramic pressure sensor typically has four thick-film resistors, which act as strain gauges and transduce a strain into an electrical signal. The sensing resistors are located on the diaphragm so that two are under tensile strain, and two are under compressive strain. These four resistors are electrically connected in a Wheatstone-bridge configuration and excited with a stabilized bridge voltage. The Wheatstone-bridge is integrated with the electronic conditioning circuit in one single ceramic substrate

In another embodiment a capacitive ceramic pressure sensor is utilized instead of or in addition to one or more of the pressure sensors described above. A capacitive ceramic pressure sensor is based on the fractional change in capacitance induced by an applied pressure. The capacitance change is due to the varying distance between the electrodes of the air-gap capacitor. These electrodes are within the cavity of the LTCC structure. The bottom electrode of the capacitor is on the rigid substrate and the upper electrode is on the deformable diaphragm. The areas of the electrodes and the distance between them define the value of the initial capacitance (C0) of the capacitive pressure sensor. The capacitive ceramic pressure sensor is preferably integrated with an electronic conditioning circuit. The power consumption of the sensing element for the capacitive ceramic pressure sensor is advantageously very low and depends mostly on the values of the operating frequency and the voltage, as well as the capacitance of the sensing element. In general, the circuit has a capacitance of around 8 pF, an operating voltage of 1 V, and an operating frequency of 10 kHz. These parameters result in a power consumption of about 0.5 μW. The power consumption can be calculated from the impedance and the applied voltage.

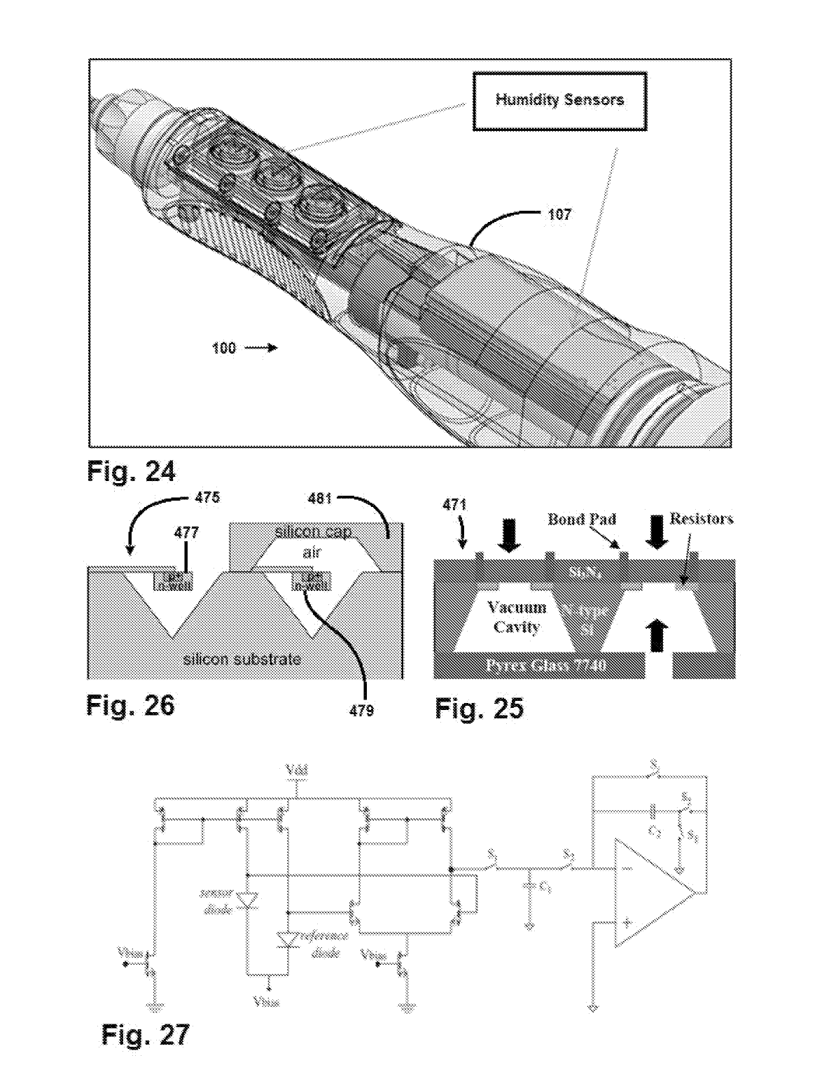

In another embodiment, as illustrated schematically in FIGS. 24-27 one or more different types of humidity sensors are provided at different locations within housing 107 for sensing changes in relative humidity (RH). For instance, polymer-based resistive humidity sensors provided between primary and secondary seals may comprise one or more thermal-based humidity sensors and may be disposed in the main chamber on the motherboard, for example. Resistive-based humidity sensors mainly use ceramics and polymers as humidity sensitive materials, including TiO₂, LiZnVO₄, MnWO₄, C₂O, and Al₂O₃. In general, ceramics have good chemical stability, high mechanical strength, and resistance to high temperature. However, they have nonlinear humidity-resistance characteristics and may not be compatible with standard IC fabrication techniques.

Humidity sensors based on polyimide piezoresistive films are particularly preferred, as they provide high sensitivity, linear response, low response time, and low power consumption. These sensors rely on humidity-dependent mechanical stress of polyimide piezoresistive film to convert changes in relative humidity into an electrical signal. Humidity sensors based on polyimide films are also robust and tolerant to standard IC fabrication techniques. This allows for integration of one or more humidity sensors with other standard integrated circuitry contained within the handpiece 100. Polymer-based resistive humidity sensors based on other humidity-sensitive dielectric materials, such as polyvinyl alcohol, phthalocyanino-silicon, and nafion may also be used with efficacy.

In another embodiment a humidity sensor based on thermal-conductivity is utilized instead of or in addition to one or more of the humidity sensors described above. As illustrated schematically in FIG. 25, one suitable embodiment of a thermal-conductivity-based humidity sensor 471 works by measuring difference between the thermal conductivity of air and that of water vapor at elevated temperatures. Heated metal resistors are provided on two different diaphragms as sensing elements, as illustrated. One diaphragm is exposed to the humid environment that causes the resistor to cool down with increased humidity, while the other one is sealed from the environment. These types of humidity sensors not only prevent condensation of water on the sensing elements but also desirably provide a linear response, low hysteresis, and long-term stability.

FIG. 26 is a schematic illustration of another embodiment of a suitable humidity sensor based on thermal conductivity. Humidity sensor 475 uses suspended diodes 477, 479 that require less power for heating while providing the required sensitivity. These humidity sensors are implemented by post-CMOS processing of CMOS fabricated chips to obtain suspended and thermally isolated diodes. This approach allows the monolithic integration of the sensor on a single substrate. The humidity sensor implemented with this approach also provides a linear response and low hysteresis. In this design, as illustrated in FIG. 26, there are two diodes—a sensor diode 477 and a reference diode 479. The reference diode 479 is sealed from the environment by attaching a silicon cap 481, while the sensor diode is exposed to the environment desired to be sensed.

Both of the diodes are suspended on a thin cantilever, as illustrated, and heated to temperatures in the range of about 250 degrees C. The thin cantilevers provide one or more conductive paths for electrically communicating with each diode 477, 479 and also provide thermal isolation from the underlying substrate. Both of the diodes are heated, for example, by applying substantially constant current in equal amounts. It takes about 0.1 mW of power to heat the diodes, which is a small portion of the total power consumption of the sensor 475 (˜1.38 mW). The exposed sensor diode 477 will have humidity-dependent thermal conductance, while the reference diode 479 will have a substantially fixed thermal conductance. Therefore, the diodes will heat up to different temperature levels, providing different diode turn-on voltages, resistance and/or break-over characteristics. By comparing the diode voltages of the reference and the sensor diodes 477, 479, it is possible to determine the humidity level.

A CMOS-based humidity sensor 475 provides a number of advantages when integrated into a handpiece in accordance with the present invention. For example, the diodes 477, 479 can be heated up with a low power, and they provide high sensitivity. As the diodes are heated up, no water condensation can occur on the sensing elements. The fabrication process is CMOS compatible and requires only a simple etching step after CMOS process, i.e., the sensor can be fabricated at low cost. Also, the signal processing circuit can be integrated with the sensor, which improves sensitivity, reduces noise, and allows for a smaller sensor package.

FIG. 27 is a schematic electrical diagram illustrating one possible embodiment of a signal processing circuit suitable for use with the humidity sensor 475 as described above. The thermal conductance of the sensor diode 477 increases with the increasing amount of water vapor, which results in a decrease of the temperature of the diode 477. Due to the negative temperature sensitivity of the diode, the output voltage of the sensor diode increases, while the output voltage of the reference diode remains constant. The difference between the diode voltages is converted into a current by a differential trans-conductance amplifier, and this current is integrated through a switched capacitor integrator in order to obtain an amplified output signal with a gain of 60 dB.

In another embodiment a gravimetric humidity sensor is utilized instead of or in addition to one or more of the humidity sensors described above. Gravimetric humidity sensors rely on sensing measurable changes in mass due to absorption of moisture. Change in mass can be detected, for example, by sensing changes in the resonant frequency of a quartz resonator such as a quartz crystal microbalance.

In another embodiment a capacitive-based humidity sensor is utilized instead of or in addition to one or more of the humidity sensors described above. Capacitive-based humidity sensors typically rely on sensing measurable changes in the capacitance of a humidity-responsive substrate. For example, a change in RH can be detected by humidity-induced changes in the dielectric constant of a thin film dielectric.

### Sensor Packaging

Those skilled in the art will appreciate that all of the various sensors described above (including motion sensors, pressure sensors, humidity sensors, and any other sensors desired to be incorporated into a handpiece) may be packaged and placed internally within the housing 107 at various locations where sensing is desired. If desired, one or more polymer gaskets with integrated conductors may be used to provide a hermetically sealed package configured to protect the sensor from harsh environments. In another embodiment additional protection may be provided in the form of a conformal non-hermetic coating configured to keep moisture away from any portion(s) of a sensor desired to be maintained dry. Preferably, the conformal non-hermetic coating displays excellent resistance to mobile ion permeation and high humidity, and has suitable thermal and mechanical properties, chemical compatibility, reasonable curing temperature, low residual stress, good adhesion, and good solvent resistance over a wide range of temperatures. For example, some or all of the aforementioned objectives can be achieved by applying a parylene coating (preferably less than 2 mm thick) to the outer body of the sensor package and any associated electrical components for which additional moisture protection is desired.

The location and mechanical securement of the various sensors within the housing 107 is preferably selected so as to avoid or minimize potentially-damaging mechanical shock and vibration, such as caused by dropping or striking the handpiece 100 against a hard surface or by sudden jolting or development of excessive vibration during handpiece operation and/or sterilization. Mechanical shock is a particularly major cause of degradation and failure in MEMS-based devices and other sensitive components.

Mechanical shock develops from a large force over a very short time interval relative to the settling time or natural decay time of an elastic body (e.g., housing 107). Typically, all large-amplitude, short-duration, impulse-like loads, such as a drop from a table, are characterized as shock. Shock loads are not easy to quantify due to their wide amplitude range (20-100000 g or larger), wide range of duration (50-6000 ρs), and largely unknown and unrepeatable “shape” (pulse, half sine, etc.). In situations such as free fall, an object experiences 1-g acceleration until it impacts a surface. When impacting a hard surface, an object may experience substantial (˜2000 g) shock when dropped from a mere 1 m (e.g., from an operating table or surgical tray).

The detrimental effects of mechanical shock on MEMS-based devices can be particularly significant. The most serious detrimental effect is immediate structural damage to the MEMS device, such as initiation and propagation of stress cracks and, in some cases, complete fracture of device structures. MEMS devices typically include delicate mechanical structures that are particularly susceptible to vibration- or shock-induced stress damage. On the other hand, it is desirable to provide a handpiece 100 having a durable construction designed to withstand normal use and abuse, including multiple drops from tabletop height, while maintaining at all times accurate motion- or gesture-based input sensing.

To avoid and minimize possible degradation due to fracture, fatigue and creep, preferably some or all of the various sensors (and particularly any MEMS-based sensors) are mounted within an internally-sealed cavity 118 (see, e.g., FIG. 15, and FIGS. 23A-23B) and resiliently supported therein so as to mechanically-isolate the sensors from any large undesired shocks and vibrations such as caused by dropping or striking the handpiece 100 against a hard surface. For example, some or all of the various sensors may be supported within a sealed or non-sealed primary enclosure 492 (e.g., a cylindrical metal enclosure). If desired, a secondary enclosure 494 may also be provided nested within the primary enclosure and mechanically isolated therefrom using memory foam or another suitable viscoelastic material 496, as illustrated in FIG. 23A.

If desired, the cavity 118 and/or any primary or secondary containment enclosures may be fully or partially filled with micro granular SiO₂ beads 490, as shown in FIGS. 23A and 23B. These may comprise, for example, SiO₂ micro-glass beads having a diameter of 68 μm and commercially available from Binex®. Most preferably, the SiO₂ micro-glass beads 490 are packed tightly into the cavity 118 and around the sensors 113, 115, 401, 475 (and/or any other sensors disposed therein) so as to substantially prevent the internal free flow of the micro granular beads while mechanically securing and protecting the sensors from potential damage cause by large shock inputs and other high-frequency mechanical excitations.

Advantageously, the closely-packed micro granular beads 490 are able to absorb and dissipate short duration mechanical shocks through micro-kinetic inter-particle and particle-to-wall collisions resulting in a safe release and dissipation of energy as friction. On the other hand, the closely-packed micro granular beads preferably do not substantially absorb or impede low or medium frequency mechanical input and vibrations which are desired to be sensed by the sensors. See, for example, S. Yoon, J. Roh and K. Kim “Woodpecker-inspired shock isolation by microgranular bed”, J. Phys. D: Appl. Phys. 42 (2009) 035501 (8pp), incorporated herein by reference in its entirety.

### Sensor-Based Control

As noted above in connection with FIG. 15, preferably one or more motion or position sensors (e.g., a 3-axis gyro 113 and/or a 3-axis accelerometer 115) are provided within the housing 107 and configured to sense various motions, positions or orientations of the handpiece 100 while the handpiece 100 is in use. Moreover, as noted above in connection with FIGS. 18 and 24, preferably one or more pressure or humidity sensors are provided within the housing 107 and configured to sense pressure and relative humidity at various locations within the handpiece 100. Advantageously, these and/or various other commercially-available sensors (e.g., tilt sensors, accelerometers, gyro sensors, impact sensors, vibration sensors, proximity sensors, temperature sensors, pressure sensors, humidity sensors, and the like) may be incorporated into the primary or auxiliary control logic of the handpiece, as discussed in more detail herein, in order to provide improved user control and/or additional user control options.

For example, using one or more sensors as described herein (e.g., gyro sensors or accelerometers), persons skilled in the art will readily appreciate and understand that a handpiece 100 can be provided that relies partially or entirely upon sensed motions or gestures for user input and control. See, for example, U.S. Pat. No. 8,286,723 to Puzio, the entire contents of which is hereby incorporated by reference. This would eliminate or reduce, for example, the need for a multi-push-button keypad, keypad seals and the leakage risks inherent in any moving component required to maintain sealed contact with another component. A motorized handpiece incorporating motion- or gesture-based controls would not only provide improved functionality and performance, but would improve durability and ability to withstand multiple cycles of sterilization and autoclaving. Those skilled in the art will appreciate that various input sensors can be disposed internally within a handpiece (e.g., in the cavity 118 or in a cavity where a keypad 125 would otherwise reside).

In accordance with one embodiment of the invention, various user input signals in the form of motions or gestures can be processed and recognized by an internal computerized control system (not shown) that computationally examines the inertial characteristics of one or more sensor data inputs and determines an overall movement or orientation of the handpiece. Based on the determined movement or orientation of the handpiece the internal control system can then control or activate various associated handpiece functions and operating characteristics. In another embodiment, the control system may simultaneously process one or more additional device-related input signals (e.g., motor speed, direction, torque, current, vibration, temperature, pressure, humidity, etc.) in order to automatically modify one or more operational characteristics of the handpiece in accordance with a predetermined or user-selected control optimization algorithm.

For example, a combination of a tri-axial accelerometer 115 and a tri-axial gyroscope 113 may be used as part of an inertial navigation system (INS) configured to calculate or estimate a relative location and orientation of a handpiece relative to a last known (or assumed) location and orientation in an inertial reference frame. Position can be estimation based on Newton's law. To give an approximate position, accelerations sensed in each of the three dimensions of free space are integrated twice starting from a known starting point. Similarly, the tri-axial gyroscope provides the sensed orientation of the handpiece relative to each dimension of free space. Changes in sensed location and/or orientation cab be used, for example, to control or adjust motor speed, or motor torque of a handpiece. For example, when the handpiece motor 103 is set for forward rotation (e.g., via a user input switch or button) the speed or output torque of the motor can be adjusted by the surgeon by rotating the hand-piece clockwise (for increased speed or torque) or counter-clockwise (for decreased speed or torque). This may be a useful control input mechanism where, for example, a surgeon desires to install a cranial screw using a driver or to remove bone burrs during a knee surgery operation using a shaver.

The speed or torque adjustment may increase or decrease linearly at a certain rate over time or, more preferably, it may be proportionate to the sensed angular rotation of the hand-piece about the axis of rotation of the working element 105 relative to a known or assumed starting position (e.g., the angular position where the working element stalls or stops rotating). For example, if the angular displacement from a stalled position is greater than +30 degrees, then maximum forward torque may be supplied. But, if the angular rotation is less +5 degrees, then the motor may provide only minimum forward torque. Similarly, if the angular displacement from a stalled position is greater than −30 degrees, then maximum reverse torque may be supplied. But, if the angular rotation is less −5 degrees, then the motor may provide only minimum reverse torque. Alternatively and/or in addition, the sensed rate of change of angular rotation may also be used to adjust motor speed and/or torque output. Rotational speed and direction can be measured in real time using conventional motor control circuitry. Reaction torque can either be measured in real time (e.g., using back EMF measurements) or it can be estimated using a pre-recorded correlation table based on measured rotational speed and motor input power, as desired.

In accordance with more sophisticated embodiments of the invention, sensed motions or gestures may include, for example and without limitation: i) full-body translational or rotational movement of the handpiece 100, ii) absolute or relative position, angle, or orientation of the handpiece in free space, iii) amount of input torque and/or force applied by a user to the handpiece, iv) amount of reaction torque and/or force applied by a user in resistance to translational or rotational movements of the handpiece, or v) tapping, jerking, shaking, or dropping the handpiece. The control system may also be configured shut off the motor 103 if an acceleration is sensed consistent with a gravitational free fall of the handpiece 100.

In accordance with one embodiment of the invention a surgical hand-piece 100 is provided that utilizes an array of sensors 113, 115, 401, 475 to sense translational and/or rotational motion of the hand-piece body in free space, as well as various operating and environmental conditions relevant to hand-piece operation and maintenance. For example, a surgical hand-piece may incorporate a wide variety of active and/or passive sensor elements, such as one or more combinations of accelerometers, gyroscopes, humidity sensors, temperature sensors, vibration sensors, pressure sensors, force sensors, torque sensors, proximity sensors, and the like. Preferably, some or all of these sensors are embedded within one or more protected enclosures that can withstand multiple cycles of human abuse and autoclaving.

An internal control system that utilizes input control data or feedback control data from one or more sensors or other signal sources can be used, for example, to achieve one or more of following objectives: i) provide a fixed or variable torque setting mimicking the function of a human wrist during a screw tightening process, ii) translate human motion or an applied torque or force into a corresponding output motion of a working element 105, or a torque or force exerted thereby, iii) provide haptic feedback such as vibration or other human-perceptible signal, and iv) modify the operating characteristics of the handpiece 100 (e.g., by precisely controlling motor 103) to enhance performance thereof and/or prevent damage thereto.

In one embodiment, a multi-sensor-based control system may be configured such that the output torque can be adjusted by the surgeon on demand simply by rotating the device like a screwdriver (e.g., clockwise to increase torque, counter-clockwise to decrease torque). More preferably, the control system of the handpiece 100 is configured such that it receives one or more user-directed control input signals from a first set of motion or position sensors (e.g., one or more gyros or accelerometers) and one or more feedback control signals from a second set of sensors and/or from one or more other feedback control signal sources so as to provide precise torque control based on sensed motions and operating conditions of the handpiece 100. A motorized handpiece 100 having such precise and intuitive control features would have particular advantage for driving bone-penetrating screws (e.g., cranial screws, spinal screws, bone fixation pins, dental implants, and other bone-penetrating screws).

The insertion cycle for a bone-penetrating screw can generally be divided into two phases: (a) the insertion phase (phase I) and (b) the tightening phase (phase II). The maximum torque that is reached during insertion is called insertional torque (IT) and the maximum torque before stripping occurs is called the stripping torque (ST). The insertional torque rises only in last few rotations, where typically a rapid increase in torque marks the beginning of the tightening phase. In practice, the torque exerted on a screw normally rises linearly as the screw is inserted, until the screw can no longer advance and the tightening phase begins. At this point, the torque is seen to rise far more rapidly, as illustrated in FIG. 29. See, for example, R. Thomas, K. Bouazza-Marouf, and G. Taylor, “Automated surgical screwdriver: automated screw placement,” Proc. IMechE Vol. 222 Part H: J. Engineering in Medicine, incorporated herein by reference in its entirety. Continuing to rotate the screw even 1-2 rotations beyond the onset of initial tightening can result in clinical failure as the torque quickly exceeds the stripping torque of the osteotomy site.

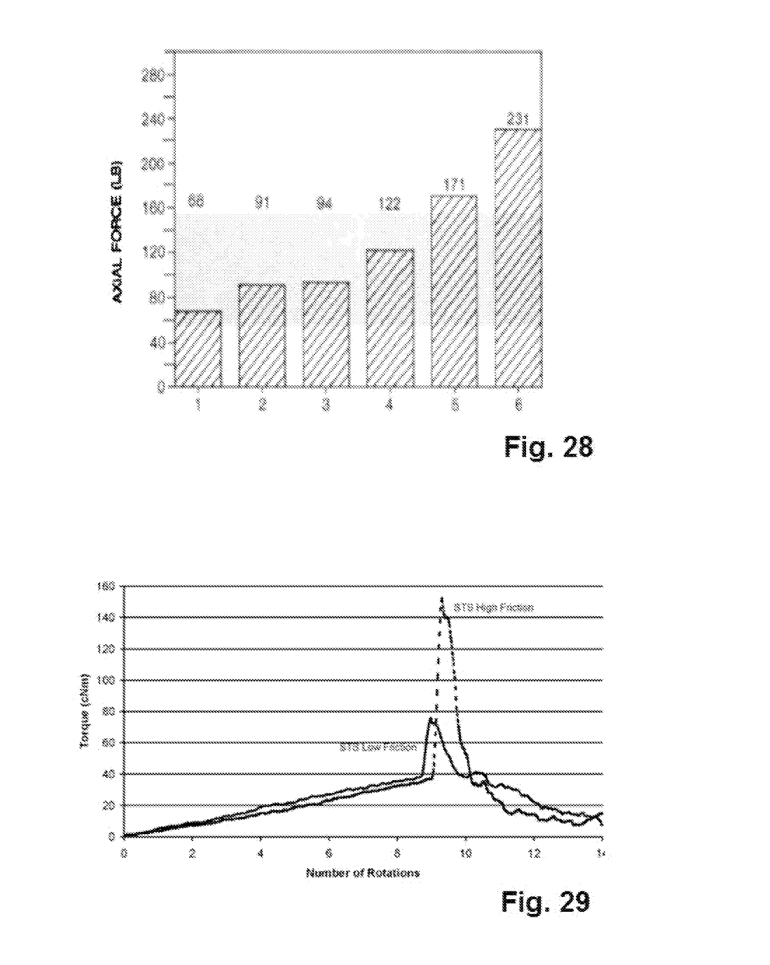

The risk of overrun and over-tightening during tapping and screw insertion is increased with the use of power tools. With a normal power screwdriver, prevention of over-tightening is entirely reliant upon the surgeon's judgment, which is based upon subtle visual and tactile information. In particular, prevention of over-tightening is dependent upon how quickly and accurately the surgeon detects the onset of tightening, both visually and from the feel of the rapid increase in torque. If detection is too late or the surgeon's reaction time is too slow, then undesirable over-tightening or stripping can occur. See, for example, Lawson, K. J. and Brems, J. “Effect of insertion torque on bone screw pullout strength,” Orthopedics, 2001, 24(5), 451-454 (finding over-tightening of bone screws resulted in approximately 40 per cent loss of pull-out strength). On the other hand, there is a competing need for increased speed of insertion while properly torquing the screw and minimizing the risk of exceeding the thread engagement strength of the bone. In addition, surgeons desire flexibility to select a desired output torque, because of personal preference, screw location, bone condition, age of patient, gender, etc. Other surgeons prefer to use a power tool to drive the screw into the bone, but prefer to seat the screw manually. Clinical studies have also shown significant inconsistencies in how much axial seating force is applied by different surgeons in identical clinical settings. For example, FIG. 28 shows a graph of measured axial load in pounds force applied by six surgeons to a bone-penetrating pin and shows a wide variation or measured results ranging from 68 pounds to 231 pounds.

When using a conventional power tool to drive a bone screw (particularly self-tapping bone screws) clinical best practices dictate that the bone screw should be initially stopped before the screw head is completely seated on the plate or bone surface, and then seated by hand and/or using a specialized torque-limiting tool in order to reduce risk of over-tightening or overrunning the osteotomy site. Most currently-available motorized handpiece or driver designs provide a single selectable torque limit beyond which the drive train will either slip or stall. This torque limit applies under all operating conditions (e.g., all motor speeds) and does not change based upon the clinical situation or the surgeon's judgment during a screw-insertion process. Thus, the surgeon must monitor and decipher the results of each separate screw insertion and make subsequent adjustments to the tool settings as required.

In accordance with one embodiment of the invention an improved motorized handpiece 100 is provided wherein a first maximum torque output is produced under a first operating condition (e.g., high motor speed) and wherein a second maximum torque output is produced under a second operating condition (e.g., low motor speed). Alternatively, the control system of the handpiece 100 may be configured such that the output torque can be instantly and easily adjusted by the surgeon while the tool is engaged with and applying torque to the bone screw.

For example, during bone screw insertion the surgeon will experience a torque reaction as the screw penetrates the bone and overcomes friction between the screw threads and the bone. This torque increases linearly until the screw head finally begins to seat against the bone plate. This happens during the last few turns as the bone plate and the screw head comes into contact. To accomplish final tightening, a general tendency by the surgeon will be to rotate the handpiece or driver in clockwise direction (intuitively, like a screwdriver) in an attempt to overcome the additional friction forces and thus fully seat the screw. But, if the motorized driver or handpiece is torque limited according to a preset or user-selected maximum torque setting (as with a conventionally-designed driver), the driver slips or stalls. Thus, to finish seating each screw the surgeon must stop and remove the motorized driver or handpiece and adjust the torque setting to supply additional torque. This process is typically repeated several times until enough torque is eventually achieved to properly seat the screw head against the bone.

In accordance with one embodiment of the present invention, control logic within the control system of the handpiece 100 is configured such that when the working element 105 (e.g., a driver blade) stops rotating during screw insertion (e.g., upon reaching an initial torque limit) and the surgeon thereafter rotates or applies torque to the hand piece in a clockwise direction (e.g., like a screwdriver) the gyroscope 113 senses the clockwise rotation of the handpiece 100 and instantly provides a signal to the control system of the handpiece 100 which instructs the motor 103 to increase the torque limit and/or deliver additional torque in increasing amounts as the surgeon continues to rotate the handpiece 100 clockwise. Thus, full screw insertion and seating can be accomplished safely and quickly using a single tool and without having to stop, remove and reset the torque setting of the tool. On the other hand, if the surgeon rotates or applies torque to the handpiece in a counter-clockwise direction then the gyroscope 113 senses the counter-clockwise rotation of the handpiece 100 and provides a signal to the control system of the handpiece 100 which instructs the motor 103 to decrease the torque limit and/or deliver reduced torque in decreasing amounts as the surgeon continues to rotate the handpiece 100 counter-clockwise.

During screw removal (e.g., when the user pushes a button to reverse motor direction) the control logic may be reversed. An initial minimum torque may be provided in a counter-clockwise direction and as the surgeon rotates or applies torque to the screwdriver in a counter clockwise direction the control system causes the delivered torque to increase. Alternatively, during screw removal (e.g., when the reverse button is pressed) the motor may provide maximum removal torque. Alternatively, for either screw insertion or removal (and, optionally, regardless of the motor direction setting), upon sensing that the working element 105 has stopped rotating, the control system may cause the motor 103 or drive shaft 104 to lock in place (either mechanically or electromagnetically) such that the working element 105 is caused to rotate in fixed relative position with the handpiece 100 (effectively operating like a conventional screw driver, with or without a torque limiting feature).

Advantageously, a control system as disclosed described above can be implemented with an internal microprocessor programmed with appropriate control logic that provides rapid and precise control of the handpiece 100, as those skilled in the art will readily appreciate and understand. This feature of the handpiece 100 provides not only improved and more consistent clinical results, but also saves significant surgeon time, because a single tool can be used to quickly drive and seat each bone screw. For example, a cranial screw only takes roughly 300 milliseconds to be fully seated. During this time a brushed DC motor must turn at 14,000 rpm in order to achieve a 300 rpm screw speed on the output of a 64:1 gear box. The final screw torque is achieved during the last ¼ to ½ turn of the screw. At 300 rpm, the screw will turn 5 rev/sec achieving its final torque within 1.5 revolutions (short low pitched screw).

Optionally, the control electronics in accordance with the present invention may be configured not only to monitor peak torque but also to predict the screw torque profile and begin to slow and reduce momentum of the motor 103 prior to reaching predicted maximum torque and final tightening. To deliver such precise output control, preferably the motor control circuit 311 (see, e.g., FIG. 8A) is further configured to dynamically break the motor 103 through, for example, an H-bridge, while simultaneously monitoring drive current and back EMF.

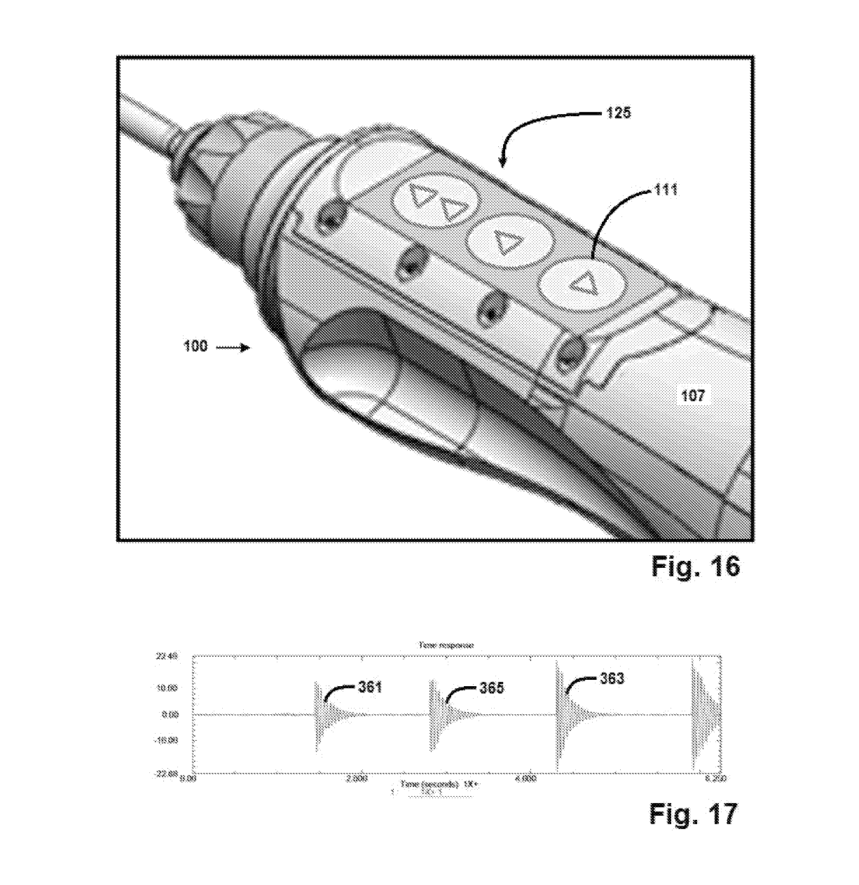

In another embodiment, one or more “virtual buttons” 111 may be provided on the outer housing 107 as engraved, screened, etched or printed icons, as illustrated in FIG. 16. Various sensors may be configured within the housing such that a user may tap on a particular virtual button 111 to selectively actuate a particular associated function (e.g., on/off, forward/reverse, faster/slower). Those skilled in the art will readily appreciate that vibration analysis and pattern recognition may be used to process sensor output data from multiple accelerometers and/or other sensor devices, to locate a precise or approximate position on the housing 107 where a user has tapped. For example, the control system may utilize various computer-learned or statistically-developed algorithms configured to determine or estimate a location of a tap by triangulation of signals or analysis of surface waves.

In another embodiment, one or more sensor signals reflective of the rigid body, bending, and twisting modes of the housing 107 are computationally analyzed and modeled in a predictive algorithm to determine or estimate a location of a sensed impulse such as a tap. Tapping intensity can also be sensed and classified, for example, as either light, medium or hard. For example, FIG. 17 shows the time domain response of an accelerometer responding to sensed tapping on the housing 107 having varying intensities. The amplitude of acceleration ranges from roughly just less than 10 m/s2 to just greater than 20 m/s2, as illustrated. Thus, for example, a tap 361 registering an acceleration response less than 10 m/s2 may be classified as a light tap. A tap 363 registering an acceleration response greater than 20 m/s2 may be classified as a hard tap. And a tap 365 registering an acceleration response between 10 and 20 m/s2 may be classified as a medium tap.

In another embodiment, haptic feedback may also be provided through, for example, a vibration, tap or click generator. For example, when a user touches a touch sensor, presses a piezoelectric button, or taps a virtual button 111 a mechanical vibration, click, or tap may be generated internally so as to provide improved user control feedback and/or more intuitive handpiece operation. In another embodiment, multiple click, tap or vibration generators may be provided and disposed in different areas of the handpiece housing 107, for example, directly under a virtual button 111. In this manner, user-control feedback such as clicks, taps, or vibration may be sensed by a user at different surfaces or areas on the housing 107. Alternatively, the motor control system may rapidly change current flow direction to the motor 103 and therefore the rotation of the motor to generate desired vibration feedback and/or other feedback. For example, the frequency and duration of the forward-reverse motor oscillation can be used to communicate control feedback (e.g., button press acknowledged), operating conditions (e.g., device on) or fault conditions (e.g., maintenance required).

### Sensor-Based Monitoring

Some or all of the various sensors disclosed and described herein and the resulting sensor data may be monitored and/or recorded both while the handpiece 100 is in use and when it is not in use (e.g., during autoclave or sterilization cycles) for purposes of providing improved performance, durability, maintenance monitoring, and failure prediction. To ensure maximum accuracy and reliability of the handpiece control system, preferably, multiple redundant sensors 113, 115, 401, 475 are preferably provided at various locations within the housing 107 in a multi-redundant fault-tolerant design. Traditional redundancy strategies include: hardware redundancy, software redundancy and analytical redundancy. In accordance with one embodiment of the present invention, hardware redundancy is employed by way of multiple redundant sensors configured to achieve a high level of fault tolerance and improved accuracy and performance. The approach is based on the assumption that measurements from the various sensor systems are independent, redundant, complementary and/or cooperative. The different control input signals (from sensors and/or other signal sources) are preferably combined by means of one or more data fusion algorithms, so that the overall system performance is better than that of each individual system acting alone.

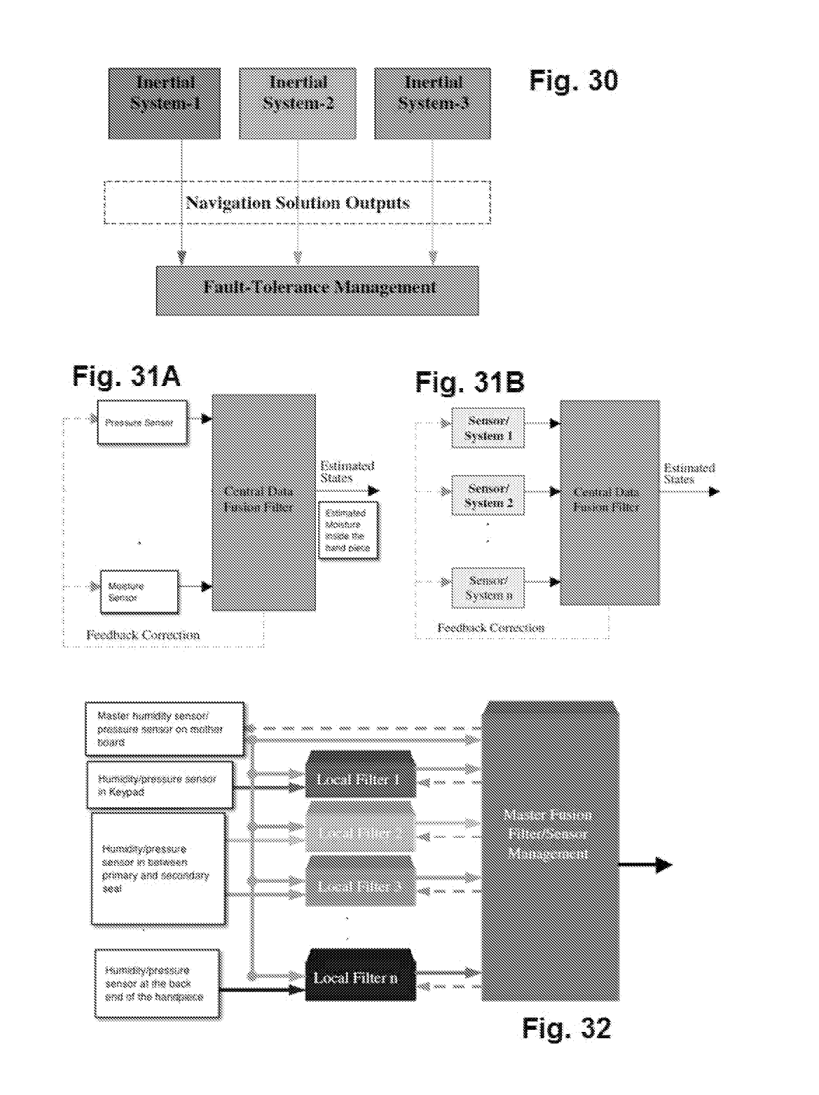

FIG. 30 is a schematic block diagram of one embodiment of a system-level redundancy architecture for an inertial navigation system in which three different sensor groups (Inertial Systems 1, 2, and 3) provide inertial input data to a fault-tolerant control management system. The Inertial Systems 1, 2 and 3 operate independently from one another so that there is preferably no data communication between these systems. This is generally known as an independent system architecture. Well-known fault-tolerant algorithms and control methods are then used to check for consistency and potential failures of any of the sensors in each of the inertial systems. For example, a majority-voting method or weighted-mean method may be used to determine which inertial system and/or which sensors are providing most accurate data. In order to achieve fail-operational/fail-safe operation, preferably at least three inertial systems are used. A key advantage of this architecture is that the design and integration is simple and does not require complex fault-tolerant methods for the diagnosis of system failures. Preferably, the handpiece 100 is configured such that, if any sensor fails, a user output signal is generated alerting the user of the faulty sensor to be repaired or replaced.

FIG. 31A is a schematic block diagram of another embodiment of a system-level redundancy architecture incorporating a central data fusion filter and feedback correction. In this fault-tolerant design multiple redundant sensors are provided and logically arranged in one or more sensor suites or arrays each configured to sense a particular condition, in this case pressure and moisture. Each sensor suite may comprise three or more sensors of the same type or a different type. Preferably, different types of sensors are used in each suite such that different strengths and weaknesses of each sensor design can be exploited and/or compensated. Again, the approach is based on the assumption that measurements from the various sensor systems are independent, redundant, complementary and/or cooperative. The different sensor input signals (from sensors and/or other signal sources) are combined by means of one or more data fusion algorithms, so that the overall system performance is better than that of each individual sensor system acting alone. Optionally, feedback from the data fusion algorithm may be used to adjust the sensor output data from one or more individual sensors so as to compensate for degradation in performance over time or poor performance under particular operating conditions (e.g., high-temperature, or heavy workload conditions). This multi-sensor redundancy architecture is a cost-effective approach that exploits the benefits of high-speed embedded microprocessor systems.

FIG. 31B is a schematic block diagram of a more generalized embodiment of a system-level redundancy architecture incorporating a central data fusion filter, feedback correction and multiple redundant sensor systems. Similar to the system-level redundancy architecture described in connection with FIG. 31A this fault-tolerant design includes multiple (up to n) different multiple-redundant sensor systems configured to sense any number of conditions desired to be sensed, including tri-axial motion, tri-axial orientation, pressure, temperature humidity, moisture, motor speed, motor torque, shaft rotation or position, back-EMF, vibration sensors, etc. Each sensor system may comprise three or more sensors of the same type or a different type. The different sensor input signals (from sensors and/or other signal sources) are combined by means of one or more data fusion algorithms, so that the overall system performance is better than that of each individual sensor system acting alone. Optionally, feedback from the data fusion algorithm may be used to adjust the sensor output data from one or more individual sensors so as to compensate for degradation in performance over time or poor performance under particular operating conditions (e.g., high-temperature, or heavy workload conditions). This multi-sensor redundancy architecture provides a more-generalized cost-effective approach that exploits the benefits of high-speed embedded microprocessor systems.

FIG. 32 is a schematic block diagram of a more sophisticated embodiment of a system-level redundancy architecture incorporating a federated data fusion filter, feedback correction and multiple redundant sensor systems. This design incorporates a two-stage filtering architecture wherein all of the parallel local filters combine their own sensor systems with a common reference system to obtain multiple local estimates of the various sensor system states. These local estimates are subsequently fused in a master filter to achieve global sensor estimations. By using a common reference system, all parallel filters have a common state vector. The federated filter is generally designed on the basis of two different design approaches. In the first approach, local filters are designed independent of the global performance of the federated filter and estimate n sets of local state vectors and their associated covariances by using their own local measurements. These n sets of the local state estimates are then weighted by their error covariances to obtain the global state estimates. The second approach is based on the global optimality of the federated filter. The local filters are derived from the global model of the federated filter and estimate n versions of the global states from local sensor measurements. These n versions of estimates are weighted by their error covariances to obtain the global optimality. The master filter is preferably a weighted least-squares estimator. This strategy allows the control system to account for various potential failure modes and overall system degradations (e.g., seal failure, motor failure, etc.) that may affect multiple groups of local sensors differently.

### Failure Prediction

Those skilled in the art will appreciate that sophisticated predictive analytics algorithms can be utilized as part of a condition monitoring system (CMS) to provide early diagnosis of problems and to help prevent unscheduled failure or shut down of the handpiece 100. Advantageously, some or all of the various sensors disclosed and described herein and the resulting sensor data may be monitored and/or recorded both while the handpiece 100 is in use and when it is not in use (e.g., during autoclave or sterilization cycles) for purposes of providing improved maintenance scheduling and failure prediction.

Maintenance strategies generally fall into two basic types: corrective maintenance and preventative maintenance. In a corrective maintenance strategy, parts are only replaced or repaired after they have failed. This means that the part's service life is fully utilized, but failure occurs at any moment, which potentially decreases the dependability and useful availability of a maintained system. A preventive maintenance strategy aims to prevent failure by replacing or repairing parts before they fail. In this way, maintenance activities can be planned at suitable moments such that they do not strongly affect the availability of the maintained system. However, since the actual moment of failure is hard to predict, many parts are replaced far before the end of their useful service life, which increases the maintenance costs considerably.

Therefore, one important aspect of a preventive maintenance strategy is determining optimal maintenance intervals for servicing, repairing and/or replacing various parts of the system. If the intervals are too long, failure will likely occur during use. On the other hand, if the intervals are too short then the service life of many parts will only be partially utilized and the amount of system down-time and labor hours required for maintenance may be unacceptably high. Further complicating the analysis is the fact that the optimal maintenance interval will not be the same for every part in the system. Some parts will wear out more quickly than others and/or require more maintenance. The criticality of certain components as determined by the failure mode (minor impairment or inconvenience versus complete system failure) will also impact the analysis.

Those skilled in the art will appreciate that there are several common approaches for determining a recommended preventative maintenance schedule and protocol. One approach often used is based on the moment in the life cycle at which specific maintenance intervals and maintenance protocols are determined. Traditionally, the manufacturer quantifies the interval during the design phase of the system or component, using assumptions on the future usage. This leads to a static maintenance program in which fixed intervals and maintenance protocols are applied during the complete service life of the system, disregarding any variations in usage.

In one embodiment of the present invention, a dynamic CMS and maintenance protocol is deployed, wherein the actual usage or system degradation is taken into account and the required maintenance intervals are regularly updated or even fully determined during the service life. According to this approach components are preferably replaced or repaired shortly after the condition of the component reaches a critical level as determined by one or more internal sensors and/or other internally developed data. In a more proactive variant, a predictive analytics model is developed based on recorded sensor and other data and multiple observed component failures across multiple devices. For example, such a model may be used to make specific component-failure predictions and recommended preventative maintenance protocols to be carried out during the remaining useful life of the handpiece 100.

While no technology can prevent normal handpiece wear and/or the need for maintenance, predictive analytics and predictive diagnostics as disclosed and described herein can be used to prevent critical system failures in a surgical environment by detecting impending problems early and allowing surgeons to send out a handpiece for maintenance and repairs prior to failure. Such predictive analytics also provides for early and actionable real-time warnings of impending handpiece failure and problems that might otherwise have gone undetected. This increases the dependability of the handpiece and allows the handpiece manufacturer to move from reactive and time-based maintenance to proactive and preventative maintenance. Handpiece manufacturers, therefore, improve their availability and reliability, increase efficiency and reduce maintenance costs.

Predictive analytics generally works by developing a unique set of failure profiles for each hand piece design across all known loads, ambient conditions, and operating contexts and failure modes. It calculates observed and/or predicted operational relationships among all relevant parameters, such as loads, temperature outside the motor housing, pressures inside the hand piece housing, vibration readings, ambient conditions and the like. It then takes recorded and/or actual real-time sensor readings and compares them to normal sensor readings that the model would predict or expect. If there are significant differences between actual and expected sensor readings then predictive diagnostics are used to identify the most likely problem.

Although there are an unlimited number of root causes for a handpiece failure, the number of failure effects that can be observed by sensors is limited. These assume that the degradation process of each considered component can be determined by different monitoring techniques (e.g. pressure sensor and/or humidity sensor monitoring for leakage and 3-D accelerometer and/or 3-D gyroscope in case of torque sensing mechanisms). Based on this degradation process, decisions (e.g. time of inspection, time of maintenance) to achieve optimal maintenance are made. However, when realistically modeling such facilities a system perspective should be taken. It is not cost effective to accommodate every component in a production machine with its specific monitoring system. Therefore, condition monitoring systems (CMS) exist which are capable of monitoring different components and failure modes simultaneously. These systems also can take into account the performance of the CMS itself, the ability to detect a failure mode and at what stage of deterioration it can be detected, and the added value of condition monitoring of a given component for a given failure mode. This determines the time to react to the potential failure of a component, which determines the ability to avoid long down times of the handpiece by the possibility of planning maintenance actions in advance.

Preventing secondary damage on other components by detecting an incipient failure is another advantage of implementing a CMS as disclosed and described herein. This benefit is dependent on the time when the CMS is capable of detecting a potential failure. The earlier the CMS detects the deterioration propagation, the less secondary damage will occur. A concept that is often used to describe the deterioration process of a component and the performance of on-condition maintenance tasks is the P-F curve and P-F interval (see, e.g., FIG. 32). A balance typically exists between the performance and cost of the CMS. This is certainly the case for critical surgical equipment such as a handpiece with a short P-F interval or long logistical waiting time (e.g. replacement motors with a lead time to obtain of 4 months or more). When the CMS only detects the failure in a late stage of the deterioration process, little or no time is left to react to the failure propagation and this can result in more costly corrective maintenance with potential secondary effects on other components. For this reason, the performance of a CMS and potential secondary damage propagation are preferably taken into account when determining the added value of implementing condition-based maintenance.

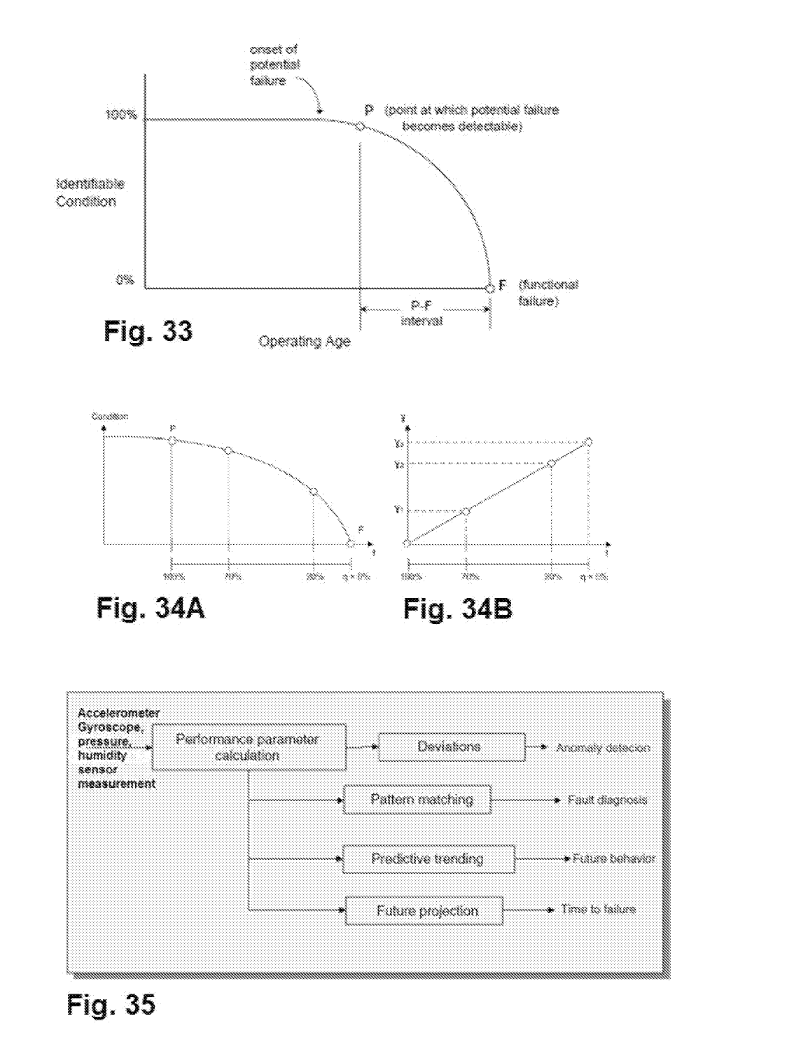

FIG. 33 shows the typical P-F (“Moubray”) curve that is used to model or predict failure patterns that can be detected by condition monitoring. This curve visualizes the deterioration in time of a particular component. When a component is operated, it will start to deteriorate until it completely loses its capability to carry out its function. The point in time where the component suffers critical failure is referred to as functional failure ‘F’. A component can perform its regular task just up to this point. The point in time where an indication of deterioration of the component can be detected is referred to as a potential failure ‘P’. The time between point P and F is called P-F interval. The central concept in this approach is the delay time of a fault, which is defined as the time lapse from when a fault could first be noticed until the time when its repair can be delayed no longer because of component failure. P-F curve, where the point in time where an indication of deterioration of the component can be detected is referred to as a potential failure ‘P’ and the point in time where the component suffers critical failure is referred to as functional failure ‘F’, is used to model the performance of a CMS on different failure modes. Consequently in this design, a system level perspective is taken.

In the context of an operating handpiece 100 potential failure (P) occurs when events lead to detectable handpiece damage that needs repair, for instance seal degradation or failure. Functional failure (F) occurs when handpiece performance no longer meets design conditions and must be shut down for repair—for instance when the hand piece housing is full of water. The curve shows that as a failure starts manifesting, the hand piece deteriorates to the point at which it can possibly be detected (P). If the failure is not detected and mitigated, it continues until a “hard” failure occurs (F). The time range between P and F, commonly called the P-F interval, is the window of opportunity during which an inspection can possibly detect the imminent failure and address it. P-F intervals can be measured in any unit associated with the exposure to the stress—in this case it is usually steam for temperature and pressure sensor and number of drops (shocks cause greatest damage to the MEMS) in case of accelerometer and gyroscope. For example, if the P-F interval is 100 autoclaves and the item will fail at 1000 autoclave, the approaching failure may begin to be detectable at 900 autoclave cycles.

Preferably, a condition-based maintenance (CBM) program is used to detect an impending failure during the P-F interval by using condition measurements, such as pressure, temperature, humidity, motion, vibration, motor performance, self-diagnostics and the like. This curve is the basis for determining an optimal time interval between two inspections in case of a CBM policy where condition monitoring is done according to fixed time intervals. Moreover, optimal maintenance actions and timing may also be determined based on the deterioration process described by the P-F curve. Besides the determination of an optimal maintenance policy, the P-F curve also gives a clear insight in the possible return on investment of a CMS. The sooner a potential failure is detected by a CMS, the smaller the component's suffered deterioration will be and depending on the P-F interval of the component an appropriate action, based on the readings of the CMS, can be carried out.

Referring to FIGS. 34A and 34B, a more formalized model of the performance of a CMS can be constructed by analyzing and comparing two interrelated parameters, γ and η where:

γ=probability of detection (%)

η=efficiency of detection (%)

The first parameter (y) represents the probability that a certain failure can be detected by a CMS. The second parameter (rt) represents the efficiency with which actual detection occurs, expressed as the fraction of the remaining time to failure divided by the P-F interval.

Both parameters are related in such a way that the probability of detection (γ) increases with time as the condition of the considered component is deteriorating. As an example, a linear relation between efficiency η and detectability y is given in FIG. 34B, however, the shape of the curve can take different forms. The exact form of this relation is defined by the CMS performance for the different monitored failure modes. In this way a direct relation between the CMS performance parameters γ and η for a given component and degree of deterioration can be defined. An efficiency of η=100% corresponds to the point on the P-F curve where an indication of deterioration can first be detected. This is referred to as potential failure or point P. An efficiency of η=0% is the point on the P-F curve where the developing failure has led to a functional failure of the component or point F on the curve. At this point any functioning of the component is impossible.

Consider, for example, a CMS where one point on the performance relation (FIG. 34B) corresponds to η=20% and γ=70%. This CMS system is on average able to detect 20% of the developing failures at 70% remaining life between P and F. When η=0% and γ=90%, this means that the CMS will miss out on 10% of the failures. In other words, in 10% of the cases a corrective action will be necessary because the CMS did not detect the developing failure. This methodology allows modeling an imperfectly performing CMS.

FIG. 35 is a schematic block diagram of a performance trending algorithm adapted for use in a motorized handpiece in accordance with another embodiment of the present invention. Performance trending is a value-added algorithm that (1) calculates numerical value for this performance using available sensor data (e.g., accelerometer, gyroscope, pressure sensor, humidity sensor, etc) and non-sensor data (e.g., motor condition, back-EMF, and related diagnostics) and (2) analyses a series of the calculated values to detect trends or shifts (anomaly detection). Recurring patterns can be archived with well-defined labels. In this case, the current pattern can be matched with one or more archived patterns to communicate an explanation for the current behavior (fault diagnosis). In differentiating between slow-moving and fast-moving trends, extrapolation of the slow trend can predict future evolution of the performance parameters. This is called predictive trending. Further, if a failed state is parameterized with respect to performance, then one can calculate how close the current state is to this failed state, and thus, estimate a remaining useful life. This functionality is called prognosis.

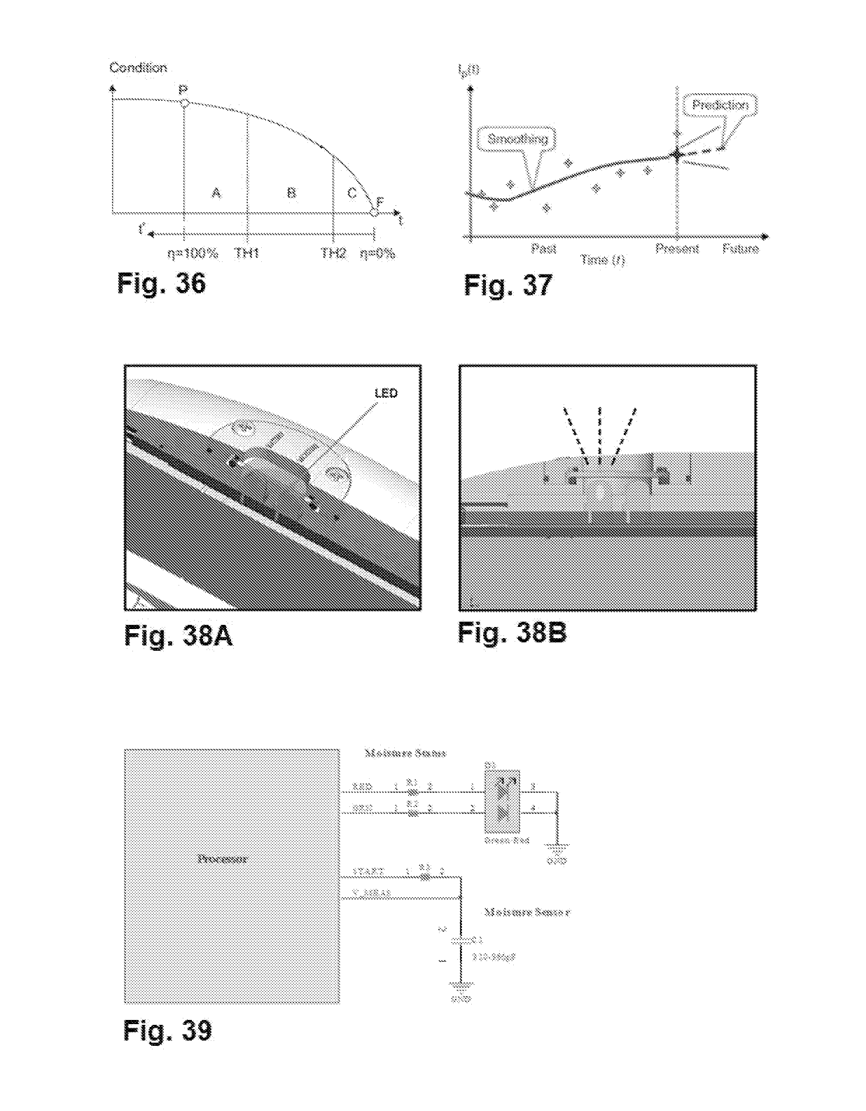

Those skilled in the art will appreciate that the CMS for the handpiece 100 may utilize any one of a number of performance trending or pattern recognition algorithms to detect one or more fault conditions that might explain a current performance parameter pattern. The first step in this analysis, as illustrated in FIGS. 36 and 37, is to determine if the currently estimated performance Ip has deviated significantly from its nominal value Ipo. Significance is established by setting up a hypothesis testing problem.

H0: Null hypothesis: (Ip-Ip0) is insignificant:

H1: Alternative hypothesis: (Ip−Ip0) is significant

When H0 is accepted, no faults are present in the hand piece. When H1 is accepted, the hand piece may be experiencing an incipient fault. Depending on the diagnostic pattern recognition, different failure modalities are recognized. These failure modalities can be plotted on a PF curve.

For example, FIG. 36 shows a P-F curve based on modeling the deterioration process of a typical handpiece over the course of its useful life. As the hand piece deteriorates, the efficiency at which electrical energy is converted to mechanical energy decreases, and thus, the hand piece performance decreases. In general, failure modes evolve from point P to point F on the P-F curve. To link the deterioration process of a failure mode with an appropriate repair or maintenance action, the P-F curve (FIG. 36) may be divided into deterioration categories. For example the P-F curve illustrated in FIG. 36 is divided in three zones A, B, and C.

Zone A defines the zone where the deterioration is in a very early stage and where the component damage (primary seals, single gyroscope, accelerometer, pressure transducer etc.) is very limited. For example, the primary seals may be damaged and/or one of the sensors may be damaged. Maintenance action may call for minor adjustments such as changing of the primary seals in order to make the component as-good-as-new or extend the lifetime of the component.

Zone B defines the zone where the deterioration and thus the component damage is significant, but no consequential damage is caused yet. For example, the secondary seals may be damaged and/or one or more of the sensors may be damaged. Maintenance action may call for repair or replacement of the specific failed components as necessary.

Zone C defines the zone where the deterioration has evolved up to the point where the component damage is maximal and consequential damage is possible. For example, none of the sensors may be working and both primary and secondary seals may be damaged. Maintenance action may call for replacement of the components such as seals and/or sensors and eventually secondary damaged components such as PCB board as necessary.

Point F defines the spot on the P-F curve where functional failure of the component (e.g., the motor) has occurred. Similar as in zone C consequential damage is possible. Replacement of the failed component and any secondary damaged components is necessary at this point.

FIG. 37 illustrates how predictive trending can be used to predict future system performance based on observed past system performance. Smoothing refers to drawing a smoothed trajectory that describes the past evolution of the performance parameters. Extending the trajectory beyond the current time is prediction. The time trajectory assumes that the performance parameters are continually evolving in an appropriate time domain (often autoclave cycles), and thus, a dynamic model can capture this evolution. Estimated performance parameters provide a useful (albeit noisy) observation for this time trajectory.

Based upon measured differences between observed and predicted performance behavior, predictive analytics is able to detect and isolates abnormal behavior. It can then shares this information with the surgeon prior to surgery via one or more haptic feedback signals or indicators.

FIGS. 38A and 38B illustrate one embodiment of a user-alert system incorporating a pair of LED indicators configured to shine through a sealed window for alerting a user to a detected fault condition. For example, a yellow light (FIG. 38B) may be used to indicate when a pressure loss has been detected between primary and secondary seals—indicating that the user should soon send the handpiece out for repairs. A red indicator may be used to indicate that serious damage has or will likely occur to the motor if the handpiece is operated—indicating that the user should not operate the handpiece and should immediately send it out for repairs. FIG. 39 is a simplified electrical schematic of one embodiment of a microprocessor-controlled user-alert system incorporating a pair of LED indicators.

### OTHER EMBODIMENTS

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary only and that the actual parameters, dimensions, materials, and/or configurations will depend upon the specific application or applications for which the inventive teachings is/are used.

Those skilled in the art will further recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed herein. While certain preferred embodiments of the present disclosure may be directed to or emphasize a specific feature, advantage, system, article, materials, kit, and/or method described herein, those skilled in the art will readily appreciate that any number of obvious combinations of two or more such features, advantages, systems, articles, materials, kits, and/or methods may be implemented and are within the inventive scope of the present disclosure. Unless specifically stated otherwise herein, any acts or steps described herein as being performed as part of a method may be performed in a sequence different than the preferred sequence described and/or may be performed simultaneously.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. The various methods or processes outlined herein may also be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

Various inventive concepts may be embodied as a computer readable storage medium (or multiple computer readable storage media) (e.g., a computer memory, one or more floppy discs, compact discs, optical discs, magnetic tapes, flash memories, flexible circuit configurations, or other tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments. Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Although this invention has been disclosed in the context of certain preferred embodiments and examples, those skilled in the art will appreciate that the present invention extends beyond the specifically disclosed embodiments to other alternative embodiments and/or uses of the invention and obvious modifications and equivalents thereof. Thus, it is intended that the scope of the present invention should not be limited by the particular preferred embodiments disclosed herein, but should be determined only by a fair reading of the claims that follow.

What is claimed is:

1. A motorized surgical instrument for driving one or more rotating and/or reciprocating working elements, said motorized surgical instrument comprising:
an outer housing;
an electric motor disposed within said outer housing;
a drive train extending through said outer housing and configured to transmit torque from said electric motor to said one or more rotating and/or reciprocating working elements; and
a sealed keypad assembly formed at least in part from a molded resilient material and comprising one or more depressible keys, wherein at least one of said depressible keys comprises a substantially resilient lower flexing portion and a substantially rigid upper cap portion and wherein said lower flexing portion is integrally molded in place with said upper cap portion.

2. The motorized surgical instrument of claim 1 wherein at least one of said depressible keys comprises one or more of: a pressure-sensitive conductive rubber, a compressible dielectric material sandwiched between one or more conductive plates, a touch sensor element configured to be activated by human touching or pressing of a finger on an outer surface thereof, or a solid-state piezoelectric switch.

3. The motorized surgical instrument of claim 1 wherein said drive train comprises one or more drive train elements configured to transmit torque or other mechanical forces by magnetic attraction or repulsion acting through a wall of a sealed housing.

4. The motorized surgical instrument of claim 1 wherein said electric motor comprises a fixed internal stator disposed within a sealed containment vessel, and an external rotor disposed outside of said sealed containment vessel and mechanically coupled to said drive train, and wherein said external rotor is configured to communicate torque from said fixed internal stator to said drive train via magnetic fields acting through said sealed containment vessel.

5. The motorized surgical instrument of claim 1 further comprising one or more motion or position sensors configured to enable a user to control some or all of the functions of the surgical instrument through one or more sensed motions or gestures, including one or more of turning, twisting, torquing, pushing or pulling, imparted by a hand of a user on said outer housing of said surgical instrument.

6. The motorized surgical instrument of claim 1 further comprising one or more humidity or pressure sensors configured to provide control or diagnostics feedback to an internal control system or performance monitoring system.

7. The motorized surgical instrument of claim 1 wherein said outer housing comprises a polymer-based composite material having a thermal conductivity higher than about 4 W/mK.

8. The motorized surgical instrument of claim 1 further comprising a thermally-conductive inner housing disposed between said outer housing and said electric motor, and wherein said inner housing is formed from CNT-enhanced copper.

9. A motorized surgical instrument for driving one or more rotating and/or reciprocating working elements, said motorized surgical instrument comprising:
an outer housing;
an electric motor disposed within said outer housing;
a drive train extending through said outer housing and configured to transmit torque from said electric motor to said one or more rotating and/or reciprocating working elements; and
one or more condition sensors configured to provide sensor output data comprising one or more operating and/or non-operating conditions of said motorized surgical instrument and a sensor monitoring system configured to use said sensor output data to provide one or more of: feedback control, performance monitoring, maintenance monitoring, or failure detection.

10. The motorized surgical instrument of claim 9 wherein at least one of said one or more condition sensors is configured to sense one or more of: heat, temperature, pressure, moisture, humidity, or leakage.

11. The motorized surgical instrument of claim 9 wherein said sensor monitoring system is configured to use said sensor output data to provide maintenance monitoring comprising using one or more algorithms to determine recommended testing, inspection or maintenance of said motorized surgical instrument.

12. The motorized surgical instrument of claim 9 further comprising a condition monitoring system configured to process said sensor output data and to recommend or enforce a maintenance protocol that takes into account the historical usage and sensed conditions of said motorized surgical instrument.

13. The motorized surgical instrument of claim 9 comprising multiple redundant condition sensors provided at different locations within said outer housing of said motorized surgical instrument and configured in a multi-redundant fault-tolerant design.

14. The motorized surgical instrument of claim 9 further comprising a user-alert system comprising one or more LED indicators configured to shine through a sealed window for alerting a user to a detected fault condition.

15. The motorized surgical instrument of claim 9 further comprising a sealed keypad assembly formed at least in part from a molded resilient material and comprising one or more depressible keys, wherein at least one of said depressible keys comprises a substantially resilient lower flexing portion and a substantially rigid upper cap portion and wherein said lower flexing portion is integrally molded in place with said upper cap portion.

16. A motorized surgical instrument for driving one or more rotating and/or reciprocating working elements, said motorized surgical instrument comprising:
an outer housing;
an electric motor disposed within said outer housing;
a drive train extending through said outer housing and configured to transmit torque from said electric motor to said one or more rotating and/or reciprocating working elements; and
one or more motion or position sensors and associated control circuitry configured to enable a user to control some or all of the functions of the surgical instrument through one or more sensed motions or gestures, including one or more of turning, twisting, torquing, pushing or pulling, imparted by a hand of a user on the surgical instrument.

17. The motorized surgical instrument of claim 16 further comprising one or more condition sensors configured to provide sensor output data comprising one or more operating and/or non-operating conditions of said motorized surgical instrument and a sensor monitoring system configured to use said sensor output data to provide one or more of: feedback control, performance monitoring, maintenance monitoring, or failure detection.

18. The motorized surgical instrument of claim 17 wherein at least one of said one or more condition sensors is configured to sense one or more of: heat, temperature, pressure, moisture, humidity, or leakage.

19. The motorized surgical instrument of claim 16 wherein said drive train comprises one or more drive train elements configured to transmit torque or other mechanical forces by magnetic attraction or repulsion acting through a wall of a sealed housing.

20. The motorized surgical instrument of claim 16 further comprising a sealed keypad assembly formed at least in part from a molded resilient material and comprising one or more depressible keys, wherein at least one of said depressible keys comprises a substantially resilient lower flexing portion and a substantially rigid upper cap portion and wherein said lower flexing portion is integrally molded in place with said upper cap portion.
